(19) United States
(12) Patent Application Publication
(10) Pub. No.: US 2026/0174543 A1
(43) Pub. Date: Jun. 25, 2026

(54) ADJUSTABLE ALL-SUTURE ANCHORING ASSEMBLY AND METHOD

(71) Applicant: Raymond Thal, Palm Beach Gardens, FL (US)

(72) Inventor: Raymond Thal, Palm Beach Gardens, FL (US)

(21) Appl. No.: 19/543,054

(22) Filed: Feb. 18, 2026

Related U.S. Application Data
(63) Continuation of application No. 18/349,523, filed on Jul. 10, 2023.

(51) Int. Cl. A61F 2/08

(52) U.S. Cl. CPC A61F 2/0805; A61F 2002/0852; A61F 2002/0888; A61F 2250/0012

(57) ABSTRACT

A method and system for secure attachment of tissue to bone and other anatomical structure is disclosed. The system includes an all-suture anchor assembly including an all-suture anchor having at least one suture strand and an all-suture anchoring element threaded along the suture strand. The system also includes an adjustable self-locking device including a length of suture passing through at least one clinching portion.

### CROSS REFERENCE TO PATENT APPLICATIONS

This application is a continuation of U.S. patent application Ser. No. 18/349,523 filed Jul. 10, 2023, entitled “ADJUSTABLE ALL-SUTURE ANCHORING ASSEMBLY AND METHOD,” which is a continuation in part of U.S. patent application Ser. No. 17/454,408, entitled “ADJUSTABLE ALL-SUTURE ANCHORING ASSEMBLY AND METHOD,” filed Nov. 10, 2021, which claims the benefit of U.S. Provisional Patent Application Ser. No. 63/113,412, entitled “ADJUSTABLE ALL-SUTURE ANCHORING ASSEMBLY AND METHOD,” filed Nov. 13, 2020, which are incorporated herein by reference.

### BACKGROUND OF THE INVENTION

### 1. Field of the Invention

The present invention relates to devices or methods used in tissue repair, more particularly, devices and methods for attachment of biological tissue (i.e., tendons or ligaments) to a bone mass.

### 2. Description of the Related Art

Soft tissues, such as tendons and ligaments, generally are attached to bone by small collagenous fibers. These connections are strong but permit the tendons and ligaments to be flexible. When a tissue, or a portion of a tissue, is torn away from the bone and requires repair, a surgeon is often required to repair the detached soft tissue with sutures, which are passed through bone tunnels and tied.

A variety of devices are available for attaching torn soft tissue to bone, such as screws, staples, cement, suture anchors. Suture anchors are commonly used to repair a torn ligament or tendon to a bone mass. These devices can be used in place of bone tunneling techniques. Suture anchors can be inserted through large open incisions or via arthroscopic surgical techniques. The arthroscope allows the surgeon to investigate a joint cavity through small incisions called portals. Various suture anchor designs are available in the market.

A suture anchor is a device that utilizes small anchors with suture materials attached thereto. A device, such as a screw, is inserted into the bone mass and anchored in place. After insertion of the anchor, the attached suture is passed through the tissue to be repaired. Recently, all-suture anchors, such as U.S. Pat. No. 9,949,733, have been developed that utilize an expandable suture or suture-based element that creates a mechanical interference under the bone surface. Sutures attached to the expandable suture element are passed through the soft tissue to facilitate the repair, in a similar fashion to the method used for repair with standard suture anchors. All-suture anchors tend to be smaller in size than standard, solid suture anchor devices. Additionally, all-suture anchors eliminate the solid anchor component that can damage joint surfaces if they inadvertently displace from the bone mass.

The tying of a knot in the suture is then required to secure the soft tissue to the bone when using either a solid suture anchor or an all-suture anchor. The process of tying a knot is time consuming and can be difficult to undertake in the tight space encountered during arthroscopic surgery and sometimes even in conventional open surgery.

Knotless anchor assemblies have been popular and are embodied in a number of prior patents such as U.S. Pat. No. 5,709,708 wherein there is provided an assembly with an anchor means having a snag means and a loop suture element attached thereto, wherein the snag means captures a loop suture element to draw tissue into secure attachment with a bone mass.

Difficulties still exist regarding the ability to adjust the tension on the repaired soft tissue. The present invention attempts to address these difficulties with a method and apparatus for knotless suture anchoring. In particular, an adjustable, knotless, all-suture anchor design is described. The present design provides for anchoring to bone using an all-suture anchor or suture-based anchor to achieve bone fixation. Tensioning of the soft-tissue repair is performed in a precise, adjustable fashion without requiring knot-tying.

### SUMMARY OF THE INVENTION

In one aspect a method is disclosed for secure attachment of tissue through application of a system comprising (1) at least one suture passing loop and (2) an adjustable self-locking device including a length of suture passing through at least one clinching portion to define a first loop member and a second loop member. The method comprises passing the at least one suture passing loop and the adjustable self-locking device through soft tissue to be secured to a bone mass, anchoring the at least one suture passing loop within a bone hole, and tensioning the adjustable self-locking device to draw the soft tissue in a controlled manner until such a time that the soft tissue is fully pulled into position.

In some embodiments the adjustable self-locking device includes a first suture passing loop linked to the first loop member and the method includes anchoring the first suture passing loop.

In some embodiments a bone anchor anchors the suture passing loop to bone.

In some embodiments an all suture anchor anchors the suture passing loop to bone.

In some embodiments a second suture passing loop is secured to the second loop member, and the method includes anchoring the second suture passing loop.

In some embodiments a bone anchor anchors the first and second suture passing loops.

In some embodiments an all suture anchor anchors the first and second suture passing loops.

In some embodiments passing the at least one suture passing loop and the adjustable self-locking device includes drawing the first loop member and the second loop member of the adjustable self-locking device through the soft tissue.

In some embodiments the step of tensioning includes pulling first and second ends of the length of suture linked to the first and second loop members in a direction away from the first and second loop members causing the length of suture to be drawn through the at least one clinching portion reducing the size of the first and second loop members and also tensioning the adjustable self-locking device to draw the soft tissue into position.

In some embodiments the method includes placing the at least one clinching portion of the adjustable self-locking device between the first loop member and the second loop member in direct contact with the soft tissue.

In a further aspect a system for secure attachment of tissue to bone and other anatomical structure is disclosed. The system includes at least one suture passing loop and an adjustable self-locking device including a length of suture passing through at least one clinching portion so as to define at least one loop member.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a schematic of a delivery inserter and all-suture anchor assembly in accordance with a first embodiment.

FIG. 2 is a schematic of an adjustable self-locking device in accordance with the first embodiment.

FIGS. 3 and 4 are schematics showing use of the first embodiment.

FIG. 5 is a schematic showing use of the first embodiment with a sleeve.

FIG. 6 is a schematic of a delivery inserter and all-suture anchor assembly in accordance with a second embodiment.

FIG. 6A is a detailed view of the section “6A” shown in FIG. 6.

FIG. 7 is a schematic of an adjustable self-locking device in accordance with the second embodiment.

FIG. 8 is schematic showing use of the second embodiment.

FIG. 9 is a schematic of a delivery inserter and all-suture anchor assembly in accordance with a third embodiment.

FIG. 9A is a detailed view of the section “9A” shown in FIG. 9.

FIG. 9B is a detailed view of the all-suture anchor including the suture strand, the all-suture anchoring assembly, and the suture strand loop. The all-suture anchor is shown in its undeployed state.

FIG. 10 is a schematic of an adjustable self-locking device in accordance with the third embodiment.

FIGS. 11 to 15 show use of the third embodiment.

FIGS. 16, 17, and 18 show use of the second embodiment in the performance of a ripstop technique.

FIG. 19 shows a technique for glenoid bone graft in accordance with the present invention.

FIG. 20 is a perspective view of an adjustable self-locking device in accordance with the fourth embodiment.

FIGS. 21, 22, and 23 show the operation of the present system.

FIGS. 24, 25, and 26 show alternate embodiments of the manner in which the fourth embodiment may be used.

### DETAILED DESCRIPTION OF THE PREFERRED EMBODIMENTS

Please add text for The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

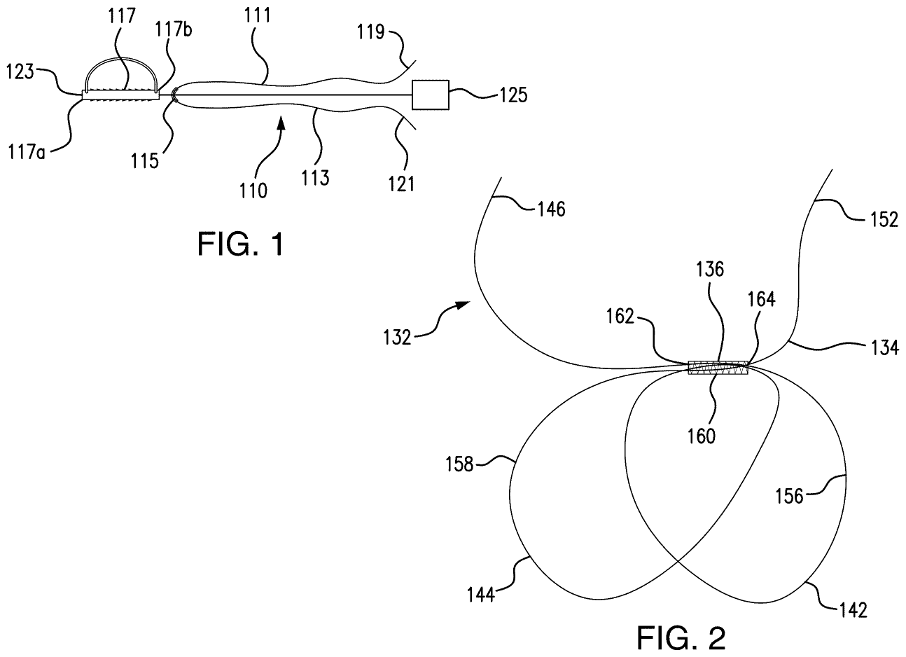

In accordance with the present invention, and with reference to FIGS. 1 to 4, a method and system for secure attachment of tissue to bone and other anatomical structure is disclosed. In accordance with a first embodiment as disclosed with reference to FIGS. 1 to 4, the system 100 includes an all-suture anchor assembly 110 and an adjustable self-locking device 132. As will be described below in detail, the all-suture anchor assembly 110 includes an all-suture anchor 111 composed of at least one suture strand 113 and an all-suture anchoring element 115 threaded along the suture strand 113. The suture strand 113 has a suture first end 119 and a suture second end 121. The all-suture anchor assembly 110 also includes a capture member 117 for controlled capture of various elements in a manner discussed below in greater detail.

The all-suture anchoring element 115 is preferably composed solely of an enlarged piece of cylindrical suture material or a suture tape. The enlarged surface area of the all-suture anchoring element 115 allows for the passage of the suture strand 113 therethrough in a manner providing for entanglement of the all-suture anchoring element 115 and the suture strand 113. The suture anchoring element 115 includes a first end and a second end, as well as a thickness, a width, and a length along a longitudinal axis.

As briefly mentioned above, the all-suture anchor assembly 110 includes the suture strand 113, which is passed through, or weaved through, the all-suture anchoring element 115 at various locations along the length of the all-suture anchoring element 115 (so as to define the all-suture anchor 111). That is, the suture strand 113 penetrates and traverses the all-suture anchoring element 115 to define apertures in the all-suture anchoring element 115. The intersections of the suture strand 113 with the all-suture anchoring element 115 are referred to herein as suture aperture locations and, as such, each of the suture aperture locations is a location where the suture strand 113 passes through the thickness of the all-suture anchoring element 115. While the suture aperture locations disclosed in accordance with a preferred embodiment are all centrally located along the all-suture anchoring element 115 so as to be oriented along the central longitudinal axis of the all-suture anchoring element 115, it is appreciated the suture aperture locations may be varied (for example, staggered on opposite sides of the central longitudinal axis of the all-suture anchoring element) without departing from the spirit of the present invention. In accordance with a preferred embodiment, the all-suture anchoring element may have various length and width dimensions depending upon the purpose for which it is intended.

It should be appreciated the above description of the preferred structure of the all-suture anchoring element 115 is as “an enlarged piece of cylindrical suture material or a suture tape”. Note that the all-suture anchoring element of some commercially available all-suture anchors have a structure that is like a sock. The suture strand is weaved through the all-suture anchoring “sock” element.

Regardless of the structure of the all-suture anchoring element, the all-suture anchoring element is inserted into the bone in an undeployed state. This undeployed state is usually “elongated.” The all-suture anchoring element is deployed (expanded) after insertion into the bone mass. The undeployed all-suture anchoring element can be inserted into the bone after a drill hole has been established or the undeployed all-suture anchoring element can be directly inserted into the bone. When the all-suture anchoring element is inserted directly into the bone, without a prior drill hole, often a “spike-tipped inserter” creates an opening in the bone for insertion of the all-suture anchoring element. Also note that there are several methods used for deployment of the all-suture anchoring element, regardless of the structure (cylindrical suture material, suture tape, or sock). Deployment can be achieved by pulling on the suture strand(s). Alternatively, a separate “deployment strand” is attached to or weaved to the all-suture anchoring element. This “deployment strand” can be pulled or tensioned to deploy the all-suture anchoring element. Additionally, mechanical systems have been developed that provide tension on the deployment suture, to deploy the all-suture anchoring element. Also note that all-suture anchor systems may have one or more suture strands. After deployment of the all-suture anchoring element, the suture strand(s) can slide within the all-suture anchoring element to facilitate knot-tying. Alternatively, the suture strand(s) may not slide after deployment of the all-suture anchoring element.

With the foregoing in mind, it is appreciated that a large variety of constructions and materials will work for the all-suture anchoring element. For example, the all-suture anchor may take a variety of forms as known in the art, for example, ConMed Y-Knot® & Y-Knot® RC, Parcus Draw Tight™, Smith & Nephew Suturefix Ultra, Smith & Nephew Q-FIX®, Zimmer Biomet: JuggerKnot® Soft Anchor; Cayenne Medical (Zimmer Biomet company) SureLock®, and Stryker® Iconix. It has been discovered that for each type of construction (i.e., braided, woven, non-woven, or knitted) it is advantageous to use a material that increases in width for every reduction in length. This advantageously provides for increased diameters for a particular number of folds, pleats, crinkles, or other changes in the shape of the all-suture anchoring element. Regardless of the material chosen for use in accordance with the present invention, the material must exhibit desirable deformation and retention characteristics.

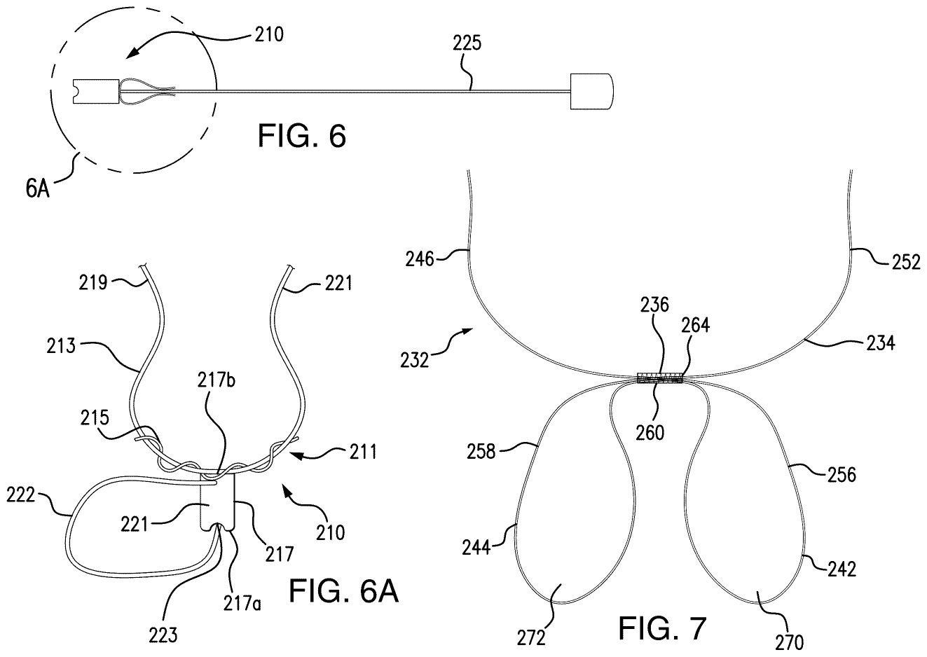

The all-suture anchor assembly 110 further includes a solid capture member 117 that is separate and distinct from the all-suture anchor 111 as disclosed in accordance with this embodiment. The capture member 117 is merely attached to the distal end of the delivery inserter 125. However, it is appreciated the capture member may be integrated with the all-suture anchor 111 as disclosed below with reference to FIGS. 6 to 8.

The capture member 117 may be composed of a variety of biocompatible materials (for example, biocompatible polymers) known to those skilled in the art. In accordance with a preferred embodiment, the solid capture member 117 includes an elongated body having a forward first end 117a and a rear second end 117b. Functionality in conjunction with the capture member 117 may be further enhanced by the provision of spikes or ridges along the outer surface thereof.

While the capture member may be integrated with or separate/distinct from the all-suture anchor, it should be appreciated the delivery and use thereof would be substantially the same.

The forward first end 117a also includes a catch member 123. In accordance with a disclosed embodiment, the catch member 123 is a lateral slot positioned at the forward first end 117a of the capture member 117. While the lateral slot 123 is shown at the forward first end 117a in accordance with the disclosed embodiment, it is appreciated the lateral slot 123 could be positioned at various locations along the capture member 117 so long as the lateral slot 123 is able to engage a suture strand loop 122 for operation in accordance with the present invention. The catch member 123 is shaped and dimensioned for capture and retention of suture(s), in particular, the suture strand loop 122, as discussed below in greater detail. While the catch member 123 is disclosed herein as being a lateral slot, it is appreciated the catch member 123 may take a variety of forms so long as it is capable of catching or snagging the suture strand loop 122, and therefore may take various shapes and have various dimensions without departing from the spirit of the present invention; for example, the catch member 123 may be shaped in the form of a hook, or other type projection, or a recess cut into the capture member 117, or a slit cut into an existing opening in the capture, for engaging the continuous loop of the suture strand loop 122.

The rear second end 117b of the capture member 117 is shaped and dimensioned for selective coupling with a distal second end of the delivery inserter 125, for example, via a threaded attachment structure. In accordance with a preferred embodiment, the delivery inserter 125 includes an elongated body with a proximal first end and a distal second end. The proximal first end is provided with a handle for actuation in accordance with the present invention. The distal second end is shaped and dimensioned for selective attachment to the capture member 117. Such delivery inserters, and in particular, compression mechanisms, are known within the art, for example, see the Draw Tight™ Suture Based Anchor System as manufactured by Parcus, and the delivery inserters of ConMed as used with Y-Knot® all-suture anchors (both of which hold the all-suture anchor inserter on a “rod-like” inserter), as well as the Smith-Nephew Q-fix inserter (that utilizes a tube-shaped inserter and the all-suture anchor is pushed out of the tube upon deployment),

The delivery inserter 125 is operated by a surgeon whereby the surgeon manipulates the all-suture anchoring element 115 and the suture strand 113. The surgeon inserts the capture member 117, the all-suture anchoring element 115, and the entangled portion of the suture strand 113 in the bone hole by pushing the distal second end of the delivery inserter 125 into the bone hole. At that point, the surgeon can push a button or turn a device on the delivery inserter 125 which enables the deployment of the all-suture anchoring element 115. While a push-button or twisting mechanism is disclosed above for deployment of the all-suture anchoring element 115, it is appreciated other known mechanisms (for example, pulling a deployment suture) for all-suture anchors may be employed without departing from the spirit of the present invention. The all-suture anchoring element 115 is then folded, bent, creased, crinkled, bunched, or otherwise changed in shape after it is inserted into the bone hole in a manner that increases the size of the all-suture anchoring element 115 in a direction substantially perpendicular to the longitudinal axis of the bone hole to develop an outwardly directed force that is directed at the walls of the bone hole. The folding, bending, creasing, crinkling, bunching, or other changes in the shape of the all-suture anchoring element 115 is achieved using known techniques.

The suture strand 113, the all-suture anchoring element 115, and the capture member 117 are installed within the bone hole in an elongated fashion to take advantage of a small diameter configuration, referred to herein as an undeployed state or installation state. Deployment occurs after the delivery inserter 125 is manipulated such that the capture member 117 captures and retains the suture strand loop 122. This capture and retention are followed by positioning of the suture strand 113, the all-suture anchoring element 115, and the capture member 117 within a bone hole for retention of the various elements to a bone mass.

In particular, the all-suture anchoring element 115 is deployed causing the all-suture anchoring element 115 to fold, bend, crease, crinkle, bunch or otherwise change shape in a manner that compresses the all-suture anchoring element 115. It is appreciated that deployment may occur before, after, or as the delivery inserter 125 is removed. As the all-suture anchoring element 115 is compressed in this manner it ultimately increases in size in a direction substantially perpendicular to the longitudinal axis of the bone hole (or otherwise oriented to contact side walls of the bone hole) and develops an outwardly directed force that is directed at the walls of the bone hole. As such, and after the delivery inserter 125, or other delivery instrument, has been removed, the all-suture anchoring element 115 exhibits an outward bias resulting in expansion of the all-suture anchoring element 115 in a direction substantially perpendicular to the longitudinal axis of the bone hole (or otherwise oriented to contact side walls of the bone hole) into which it is positioned, resulting in frictional engagement or gripping the wall of the bone, which is referred to herein as the expanded deployed state.

The all-suture anchoring element 115 is folded or otherwise compressed to form pleats between adjacent suture aperture locations. This pleating reduces the distance between the first suture aperture location and the second suture aperture location, as measured along the length of the all-suture anchoring element 115. These pleats form a bunched mass of suture material effectively increasing a diameter, or cross-sectional dimension, (as measured in relation to the axis of the bone hole) of the all-suture anchoring element 115, which ultimately causes the all-suture anchor assembly 110 to displace cancellous bone or lock beneath the bone cortex. The relative increase in the cross-sectional size of the all-suture anchoring element 115 in the direction substantially perpendicular to the longitudinal axis of the bone hole (or in another direction to facilitate contact of the all-suture anchoring element 115 with the side walls of the bone hole) creates a retention force of the all-suture anchor assembly 110. Additional retention force can be achieved when the all-suture anchor assembly is deployed beneath the bone cortex.

It is appreciated that a mechanical tensioning mechanism, as is well known to those skilled in the art, may be used during the deployment of the all-suture anchoring element 115. Such mechanical tensioning mechanisms pull or ratchet the suture while the delivery inserter 125 holds the all-suture anchoring element 115 in place. Mechanical tensioning, of this nature, may be preferable as this can more tightly ‘fold’ or ‘bunch’ the all-suture anchoring element 115, thereby increasing the created tension, that is, the outward force of the all-suture anchoring element 115, which is relative to the longitudinal axis of the bone hole and toward the walls of the bone hole, resulting from increased compression of the all-suture anchoring element 115. The changed shape of the all-suture anchoring element 115 provides security within the bone, for example, below the cortical layer.

It should be appreciated that the relative fit of the all-suture anchor 111 in the bone hole in its deployed configuration is shown as being relatively “loose.” This is done to provide a clear view for the elements making up the present invention. In practice, it is appreciated that the suture, all-suture anchoring element 115, the capture member 117, a portion of the suture strand 113, and delivery inserter 125 would be tightly pressed into the bone hole, as any excess space would need to be taken up by the expansion of the all-suture anchor 111 in a direction substantially perpendicular to the longitudinal axis of the bone hole (or otherwise oriented to contact side walls of the bone hole).

The present invention achieves secure, tensioned attachment of soft tissue to a bone mass using the all-suture anchor assembly 110 as described above in conjunction with the adjustable self-locking device 132 composed of a length of suture 134 passing through one or more clinching portion(s) 136. in such a way that first and second suture loop members 142, 144 are created.

With reference to FIG. 2, a preformed adjustable self-locking device 132 is disclosed. The adjustable self-locking device 132 is preformed to include a double loop configuration having first and second loop members 142, 144 that each traverse a path from one end of the clinching portion 136 to the other end thereof. The adjustable self-locking device 132 is formed by passing the first end 146 of the suture through the clinching portion 136 and similarly passing the second end 152 through the clinching portion 136. In various aspects, the passageway defined by the clinching portion 136 is formed during a braiding process.

Passing ends 146, 152 through the passageway defined by the clinching portion 136 forms the first and second loop members 142, 144. The first and second loop members 142, 144 define mounting portions 156, 158 of the adjustable self-locking device 132 and can be disposed generally opposite from the clinching portion 136.

The longitudinal and parallel placement of the first and second ends 146, 152 of the suture 134 within the clinching portion 136 resists the reverse relative movement of the first and second ends 146, 152 of the adjustable self-locking device 132 once it is tightened. The tensioning of the first and second ends 146, 152 causes reciprocal movement of the first and second loop members 142, 144 relative to clinching portion 136. Upon applying tension to the first and second ends 146, 152, the first and second loop members 142, 144 are reduced to a desired size or placed in a desired tension. Tension in the first and second loop members 142, 144 causes the body of the suture defining the clinching portion 136 to be placed in tension and therefore cause the clinching portion 136 to constrict about the portions 162, 164 of the suture 134 passing through the passageway defined by the clinching portion 136. This constriction causes the adjustable self-locking device 132 to “automatically” lock in a reduced size or smaller diameter configuration for the two loop members 142, 144.

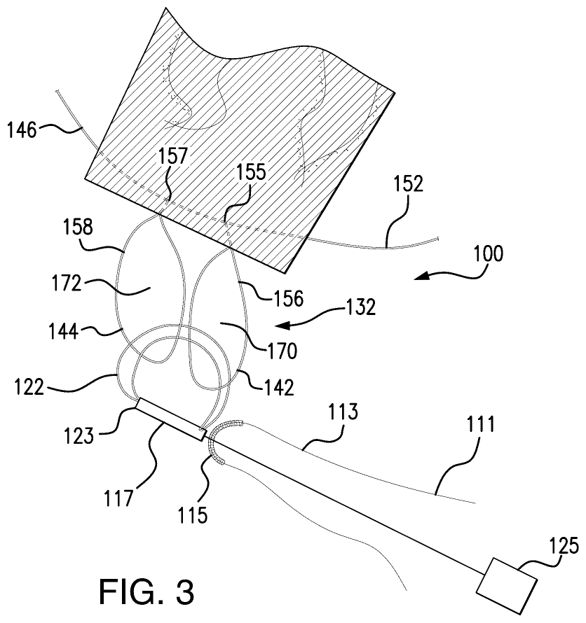

In accordance with the present method, the adjustable self-locking device 132 is first passed through the soft tissue one wishes to secure to a bone mass. Referring to FIG. 3, the adjustable self-locking device 132, in particular, the first loop member 142 thereof is drawn through the soft tissue with a surgical needle. A variety of suture passing methods can be used such as arthroscopic suture passers, arthroscopic suture shuttling devices or suture, or the like. In particular, the first loop member 142 and second loop member 144 are pulled through the soft tissue until the two mounting portions 156, 158 defined by the first and second loop members 142, 144 are of substantially the same size or different sizes and in alignment. The second loop member 144 is passed through the soft tissue at a second location 127. In particular, the first loop member 142 and second loop member 144 are pulled through the soft tissue until the two mounting portions 156, 158 defined by the first and second loop members 142, 144 are of substantially the same size and in alignment. Pulling the first and second loop members 142, 144 through the tissue is preferably achieved using a “utility suture” in a manner known to those skilled in the art. It is appreciated the first and second loop members 142, 144 are relatively long so that the opposed mounting portions 156, 158 may be brought outside of the joint under repair. As shown in FIGS. 3, the first and second end 146, 152 of the suture 134, which function as tension members as will be discussed below in greater detail, of the adjustable self-locking device 132 also extends from one of the first and second loop members 142, 144 and are similarly accessible from outside of the joint under repair. (Note that this self-locking device 132 can have just one loop member 142, without a second loop member 144. Alternatively, the self-locking device can have more than 2 adjustable loops. When using the described self-locking device (with 2 loop members 142 & 144), an alternative method would be to only pass one loop member (i.e., loop member 142) and not pass loop member 144 through the soft tissue. Both loops would still be captured by capture member 117.

With the first and second loop members 142, 144 of the adjustable self-locking device 132 outside of the joint, and with reference to FIG. 3, the suture strand loop 122 of the capture member 117 is passed through the openings 170, 172 respectively defined by the opposed first and second loop members 142, 144. That is, the free end of the suture strand loop 122 is drawn through the openings 170, 172 such that the suture strand loop 122 is intertwined or linked with the opposed first and second loop members 142, 144. In this arrangement, the clinching portion 136 of the adjustable self-locking device 132 between the first and second loop members 142, 144 is in direct contact with the soft tissue securing the suture strand loop 122 to the soft tissue such that the first and second loop members 142, 144 may simultaneously pull against the suture strand loop 122 without fear that the adjustable self-locking device 132 will become disengaged with the soft tissue. Alternatively, the first and second loop members 142, 144 can be captured by independent all-suture anchor assemblies 110 and secured to separate locations on the bone.

Thereafter, the free end of the suture strand loop 122 is captured by the catch member 123 of the capture member 117; that is, the free end of the suture strand loop 122 is captured, entangled, coupled to, or otherwise attached to the catch member 123 at the first end of the capture member 117 for manipulation of the suture strand loop 122 and ultimately fixed attachment of the free end of the suture strand loop 122 to the catch member 123 at the first end of the capture member 117 upon deployment of the all-suture anchor assembly 110 within the bone mass. It is appreciated that if the catch member 123 is not positioned at the first end of the capture member 117, the free end of the suture strand loop 122 would be fixed wherever the catch member 123 is located.

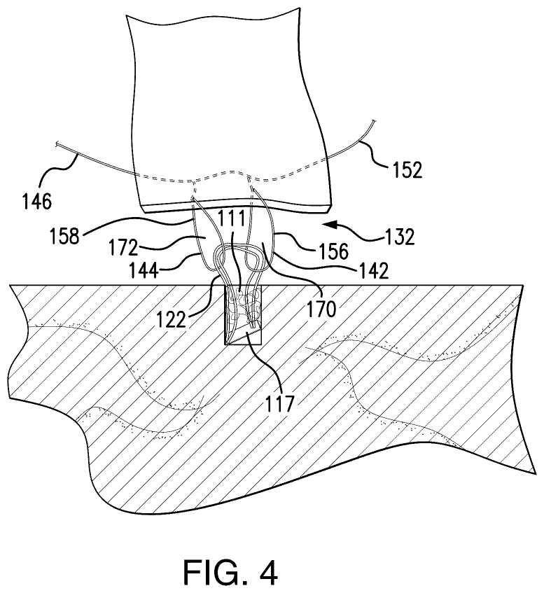

With the suture strand loop 122 passed through the openings 170, 172 defined by the opposed first and second loop members 142, 144 and the free end of the suture strand loop 122 captured by the catch member 123 at the first end of the capture member 117, the suture strand loop 122 is linked to the first and second loop members 142, 144 and ultimately the soft tissue. Referring to FIG. 4, the all-suture anchor assembly 110 is then inserted within an anchor hole preferably predrilled in the bone mass. The provision of the clinching portion 136 with the suture portions 162, 164 therethrough and under the control of the first and second ends 146, 152 of the suture 134, provides for adjustment in the size of the first and second loop members 142, 144. This adjustability is useful in several aspects of the surgical procedure.

With the all-suture anchor assembly 110 securely held within the drill hole, the first and second ends 146, 152 of the suture 134 are pulled in a manner reducing the sizes of the first and second loop members 142, 144, and also tensioning the adjustable self-locking device 132, thereby drawing the soft tissue toward the bone mass in a controlled manner. It should be appreciated the ability to reduce the size of the first and second loop members 142, 144 relative to the suture strand loop 122 is permitted as a result of the fact that the engagement point for the first and second loop members 142, 144, and the suture strand loop 122 sits above the bone hole or within the bone hole, but above the deployed all-suture anchoring element 115 and there is no impediment to the free movement of the first and second loop members 142, 144 relative to the suture strand loop 122.

In particular, the first and second ends 146, 152 are pulled in a direction away from the first and second loop members 142, 144 causing the suture 134 to be drawn through the adjustable self-locking device 132 reducing the size of the first and second loop members 142, 144, and also tensioning the adjustable self-locking device 132 to lock the suture portions 162, 164 therein, and consequently drawing the soft tissue toward the bone mass since the size of the suture strand loop 122 is fixed. The first and second ends 146, 152 of the suture 134 are pulled and the sizes of the first and second loop members 142, 144 are reduced until such a time that the soft tissue is fully pulled toward the bone mass. The procedure may be repeated to provide the desired degree of tension on the repair. The excess suture material of the first and second ends 146, 152 of the suture 134 may then be cut away and the incision closed.

Referring to FIG. 4, it is also appreciated that the present invention allows for anchoring and soft tissue attachment with limited regard for the depth of the drilled hole or the depth of the all-suture anchor assembly 110 within the bone mass. This results from the ability to draw the soft tissue toward the all-suture anchor assembly 110 and bone mass under the control of the adjustable self-locking device 132, in particular pulling of the first and second ends 146, 152 of the suture 134 which results in a reduction in the size of the loop members 142, 144. Because the size of the loop members 142, 144 dictate how close the soft tissue is pulled toward the bone mass/bone anchor, a medical practitioner can readily control the position of the soft tissue relative to the bone mass/bone anchor and control the tension applied to the repair.

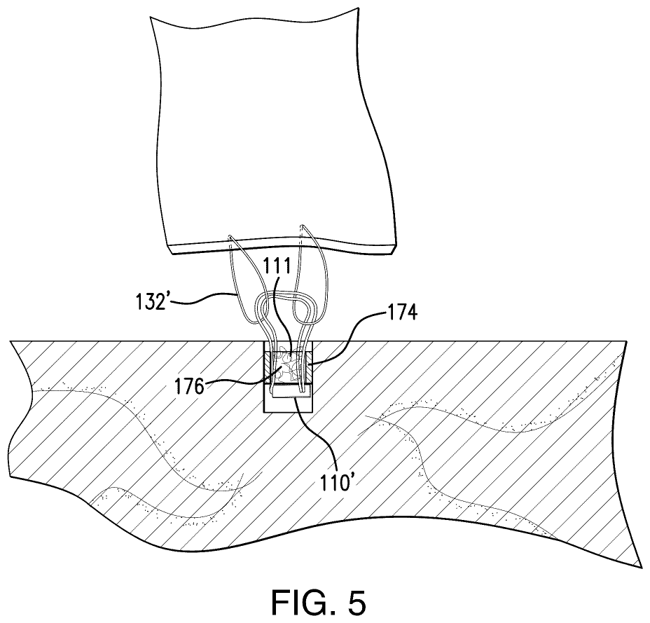

With reference to FIG. 5, an alternate embodiment is disclosed that is similar to that disclosed with reference to FIGS. 1 to 4. In the alternate embodiment, a cylindrical anchor mounting sleeve 174 is employed for secured attachment of the all-suture anchor assembly 110. The distal end of the cylindrical anchor mounting sleeve 174 is open, allowing access to the internal bone mass. Any fixation method can be utilized to affix the anchor mounting sleeve to the bone mass. In fact, standard suture anchors, and insertion methods, are known for deployment of a hollow, threaded sleeve, with attached sutures. For example, the DePuy/Synthes—Mitek Healix suture anchor or the Smith & Nephew Healicoil suture anchor both consist of a cylindrical, threaded anchor body with attached suture. These cylindrical anchors, without the attached suture, could function as the anchor mounting sleeve. It is appreciated the open cylindrical anchor mounting sleeve 174 provides an anchor recess (or anchor passageway 176 in accordance with such an embodiment) extending from the proximal end of the open cylindrical anchor mounting sleeve 174 to the distal end of the open cylindrical anchor mounting sleeve 174.

The anchor passageway 176, in the open cylindrical anchor mounting sleeve 174, allows for capture of the all-suture anchor assembly 110 on the distal end of the open cylindrical anchor mounting sleeve 174 within a small cavity defined by the bottom of the anchor hole and the distal end of the open cylindrical anchor mounting sleeve 174. The all-suture anchor assembly 110 is passed through the anchor passageway 176 with the longitudinal axis of the all-suture anchor assembly 110 aligned substantially parallel with the longitudinal axis of the open cylindrical anchor mounting sleeve 174 (see FIG. 5). Once the all-suture anchor assembly 110 has passed fully through the anchor passageway 176 and distal to the end of the open cylindrical anchor mounting sleeve 174, the all-suture anchor assembly 110 is deployed as described above to thereby lock the all-suture anchor assembly 110 in position due to the fact the length of the all-suture anchor assembly 110 is greater than the diameter of the anchor passageway 176. The fixation method of the cylindrical anchor mounting sleeve (i.e.—external threads) provides secure bone fixation. It is also appreciated, the internal structure of the bone mass is not very dense and the all-suture anchor assembly 110 may actually be pushed into, and manipulated within the bone mass for orientation, thereby obviating the need for a deep hole or a hole extending below the distal end of the open cylindrical anchor mounting sleeve 174.

Once the all-suture anchor assembly 110 is locked in position distal to the anchor mounting sleeve 174, the adjustable self-locking device 132 may be tightened as discussed above with regard to FIGS. 1 to 4.

As mentioned above, the capture member and the all-suture anchor may be constructed in an integral manner. Such an embodiment is disclosed in FIGS. 6 to 8. In particular, the all-suture anchor assembly 210 includes an all-suture anchor 211 composed of at least one suture strand 213 and an all-suture anchoring element 215 threaded along the suture strand 213 in manner discussed above with reference to the embodiment disclosed in FIGS. 1 to 4. The all-suture anchor assembly 210 also includes a capture member 217 integrated with the at least one suture strand 213 and/or the all-suture anchoring element 215 for controlled capture of various elements in a manner discussed below in greater detail. The suture strand 213 has a suture first end 219 and a suture second end 221.

The all-suture anchoring element 215 is preferably composed solely of an enlarged piece of cylindrical suture material, a suture tape, or suture sock. The enlarged surface area of the all-suture anchoring element 215 allows for the passage of the suture strand 213 therethrough in a manner providing for entanglement of the all-suture anchoring element 215 and the suture strand 213. As mentioned above, and as well appreciated by those skilled in the art, a large variety of constructions and materials will work for the all-suture anchoring element. For example, the all-suture anchor may take a variety of forms as known in the art, for example, ConMed Y-Knot® & Y-Knot® RC, Parcus Draw Tight™, Smith & Nephew Suturefix Ultra, Smith & Nephew Q-FIX®, Zimmer Biomet: JuggerKnot® Soft Anchor; Cayenne Medical (Zimmer Biomet company) SureLock®, and Stryker® Iconix.

The all-suture anchor assembly 210 further includes the solid capture member 217 that is integrated onto the all-suture anchoring element 215. Those skilled in the art will appreciate, the capture member 217 may be composed of a variety of biocompatible materials (for example, biocompatible polymers) known to those skilled in the art. In accordance with a preferred embodiment, the solid capture member 217 includes an elongated body having a forward first end 217a and a rear second end 217b. The capture member 217 is preferably secured to the all-suture anchoring element 215 by passing the all-suture anchoring element 215 through a lateral aperture formed in the capture member 217. While FIGS. 6 to 8 disclose the all-suture anchoring element 215 passing through the capture member 217, it is appreciated other mechanisms for integrating the capture member 217 with the suture strand 213 and/or the all-suture anchoring element 215 may be employed. For example, the suture strand might be passed through the lateral aperture, or both the suture strand and the all-suture anchoring element may be passed through the lateral aperture. Functionality in conjunction with the capture member 217 may be further enhanced by the provision of spikes or ridges along the outer surface thereof. As mentioned above, delivery and use of the suture strand 213, the all-suture anchoring element 215, and the capture member 217 would be substantially the same as the attached version and only the attached version will be described below in detail.

The forward first end 217a of the capture member 217 also includes a catch member 223. In accordance with a disclosed embodiment, the catch member 223 is a lateral slot positioned at the forward first end 217a of the capture member 217. While the lateral slot 223 is shown in accordance with the disclosed embodiment, it is appreciated the lateral slot 223 could be positioned at various locations along the capture member 217 so long as the lateral slot 223 is able to engage the suture strand loop 222 for operation in accordance with the present invention. The catch member 223 is shaped and dimensioned for capture and retention of suture(s), in particular, a suture strand loop 222, as discussed below in greater detail. While the catch member 223 is disclosed herein as being a lateral slot, it is appreciated the catch member 223 may take a variety of forms so long as it is capable of catching or snagging the suture strand loop 222, and therefore may take various shapes and have various dimensions without departing from the spirit of the present invention; for example, the catch member 223 may be shaped in the form of a hook, or other type projection, or a recess cut into the capture member 217, or a slit cut into an existing opening in the capture, for engaging the continuous loop of a suture strand loop 222.

The rear second end 217b of the capture member 217 is shaped and dimensioned for selective coupling with a distal second end of the delivery inserter 225, for example, via a threaded attachment structure. In accordance with a preferred embodiment, the delivery inserter 225 includes an elongated body with a proximal first end and a distal second end. The proximal first end is provided with a handle for actuation in accordance with the present invention. The distal second end is shaped and dimensioned for selective attachment to capture member 217. Such delivery inserters, and in particular, compression mechanisms are known within the art, for example, see the Draw Tight™ Suture Based Anchor System as manufactured by Parcus and the delivery inserters of ConMed as used with Y-Knot® all-suture anchors, and various compression mechanisms may be used within the spirit of the present invention.

The delivery inserter 225 is operated by a surgeon whereby the surgeon manipulates the all-suture anchoring element 215 and the suture strand 213. The surgeon inserts the capture member 217, the all-suture anchoring element 215, and the entangled portion of the suture strand 213 in the bone hole by pushing the distal second end of the delivery inserter 225 into the bone hole. At that point, the surgeon can push a button or turn a device on the delivery inserter 225 which enables the deployment of the all-suture anchoring element 215 (different methods of deployment are discussed above). While a push-button or twisting mechanism is disclosed above for deployment of the all-suture anchoring element 215, it is appreciated other known mechanisms (for example, pulling a deployment suture) for all-suture anchors may be employed without departing from the spirit of the present invention. The all-suture anchoring element 215 is then folded, bent, creased, crinkled, bunched or otherwise changed in shape after it is inserted into the bone hole in a manner that increases the size of the all-suture anchoring element 215 in a direction substantially perpendicular to the longitudinal axis of the bone hole to develop an outwardly directed forced that is directed at the walls of the bone hole. The folding, bending, creasing, crinkling, bunching or other changes in the shape of the all-suture anchoring element 215 is achieved using known techniques.

The suture strand 213, the all-suture anchoring element 215, and the capture member 217 are installed within the bone hole in an elongated fashion to take advantage of a small diameter configuration, referred to herein as an undeployed state or installation state. Deployment occurs after the delivery inserter 225 is manipulated such that the capture member 217 captures and retains suture strand loop 222. This capture and retention are followed by positioning of the suture strand 213, the all-suture anchoring element 215, and the capture member 217 within a bone hole for retention of the various elements to a bone mass.

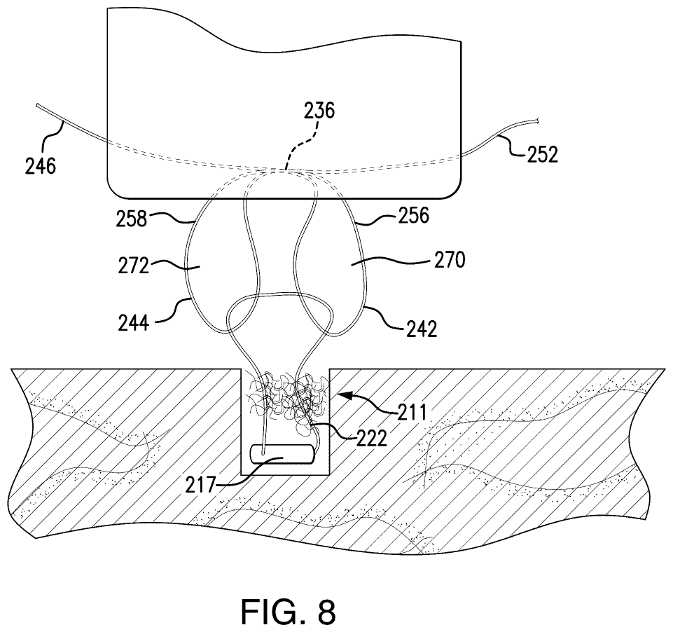

In particular, and as disclosed with the embodiment of FIG. 8, the all-suture anchoring element 215 is deployed causing the all-suture anchoring element 215 to fold, bend, crease, crinkle, bunch or otherwise change shape in a manner that compresses the all-suture anchoring element 215. It is appreciated that deployment may occur before, after, or as the delivery inserter 225 is removed. As the all-suture anchoring element 215 is compressed in this manner it ultimately increases in size in a direction substantially perpendicular to the longitudinal axis of the bone hole (or otherwise oriented to contact side walls of the bone hole) and develops an outwardly directed force that is directed at the walls of the bone hole. As such, and after the delivery inserter 225, or other delivery instrument, has been removed, the all-suture anchoring element 215 exhibits an outward bias resulting in expansion of the all-suture anchoring element 215 in a direction substantially perpendicular to the longitudinal axis of the bone hole (or otherwise oriented to contact side walls of the bone hole) into which it is positioned, resulting in frictional engagement or gripping the wall of the bone, which is referred to herein as the expanded deployed state.

As with the prior embodiment, the present invention achieves secure, tensioned attachment of soft tissue to a bone mass using the all-suture anchor assembly 210 as described above in conjunction with an adjustable self-locking device 232 composed of a length of suture 234 passing through a clinching portion 236 in such a way that first and second suture loop members 242, 244 are created. The adjustable self-locking device 232 is the same as that disclosed above with reference to FIGS. 1 to 4.

With reference to FIG. 7, the adjustable self-locking device 232 of this embodiment is preformed to include a double loop configuration having first and second loop members 242, 244 that each traverse a path from one end of the clinching portion 236 to the other end thereof. The adjustable self-locking device 232 is formed by passing the first end 246 of the suture through the clinching portion 236 and similarly passing the second end 252 through the clinching portion 236. In various aspects, the passageway defined by the clinching portion 236 is formed during a braiding process.

Passing ends 246, 252 through the passageway defined by the clinching portion 236 forms the first and second loop members 242, 244. The first and second loop members 242, 244 define mounting portions 256, 258 of the adjustable self-locking device 232 and can be disposed generally opposite from the clinching portion 236.

The longitudinal and parallel placement of the first and second ends 246, 252 of the suture 234 within the clinching portion 236 resists the reverse relative movement of the first and second ends 246, 252 of the adjustable self-locking device 232 once it is tightened. The tensioning of the first and second ends 246, 252 causes reciprocal movement of the first and second loop members 242, 244 relative to clinching portion 236. Upon applying tension to the first and second ends 246, 252, the first and second loop members 242, 244 are reduced to a desired size or placed in a desired tension. Tension in the first and second loop members 242, 244 causes the body of the suture defining the clinching portion 236 to be placed in tension and therefore cause the clinching portion 236 to constrict about the portions 262, 264 of the suture 234 passing through the passageway defined by the clinching portion 236. This constriction causes the adjustable self-locking device 232 to “automatically” lock in a reduced size or smaller diameter configuration.

In accordance with the present method, the adjustable self-locking device 232 is first passed through the soft tissue one wishes to secure to a bone mass. Referring to FIG. 7, the adjustable self-locking device 232, in particular, the first loop member 242 thereof is drawn through the soft tissue with a surgical needle. The second loop member 244 is passed through the soft tissue. In particular, the first loop member 242 and second loop member 244 are pulled through the soft tissue until the two mounting portions 256, 258 defined by the first and second loop members 242, 244 are of substantially the same size and in alignment. As shown in FIG. 7, the first and second end 246, 252 of the suture 234, which function as tension members as will be discussed below in greater detail, of the adjustable self-locking device 232 also extends from one of the first and second loop members 242, 244 and are similarly accessible from outside of the joint under repair.

With the first and second loop members 242, 244 of the adjustable self-locking device 232 outside of the joint, and with reference to FIG. 8, the suture strand loop 222 of the capture member 217 passed through the openings 270, 272 respectively defined by the opposed first and second loop members 242, 244. That is, the free end of the suture strand loop 222 is drawn through the openings 270, 272 such that the suture strand loop 222 is intertwined or linked with the opposed first and second loop members 242, 244. (As described above, the first and second loop members 242, 244 can be linked to two separate all-suture anchor assemblies 210 and attached to bone in two separate locations). In this arrangement, the clinching portion 236 of the adjustable self-locking device 232 between the first and second loop members 242, 244 is in direct contact with the soft tissue securing the suture strand loop 222 to the soft tissue such that the first and second loop members 242, 244 may simultaneously pull against the suture strand loop 222 without fear that the adjustable self-locking device 232 will become disengaged with the soft tissue.

Thereafter, the free end of the suture strand loop 222 is captured by the catch member 223 of the capture member 217; that is, the free end of the suture strand loop 222 is captured, entangled, coupled to, or otherwise attached to the catch member 223 at the first end of the capture member 217 for manipulation of the suture strand loop 222 and ultimately fixed attachment of the free end of the suture strand loop 222 to the catch member 223 at the first end of the capture member 217 upon deployment of the all-suture anchor assembly 210 within the bone mass to which the soft tissue is secured. It is appreciated that if the catch member 223 is not positioned at the first end of the capture member 217 anchor member 214, the free end of the suture strand loop 222 would be fixed wherever the catch member 223 is located.

With the suture strand loop 222 passed through the openings 270, 272 defined by the opposed first and second loop members 242, 244 and the free end of the suture strand loop 222 captured by the catch member 223 at the first end of the capture member 217, the suture strand loop 222 is linked to the first and second loop members 242, 244 and ultimately the soft tissue. Referring to FIG. 8, the all-suture anchor assembly 210 is then inserted into the bone, either directly or within a predrilled hole in the bone mass. The provision of the clinching portion 236 with the suture portions 262, 264 therethrough and under the control of the first and second ends 246, 252 of the suture 234, adjustment in the size of the first and second loop members 242, 244 provided for. This adjustability is useful in several aspects of the surgical procedure.

With the all-suture anchor assembly 210 securely held within the drill hole, the first and second ends 246, 252 of the suture 234 are pulled in a manner reducing the sizes of the first and second loop members 242, 244, and also tensioning the adjustable self-locking device 232, thereby drawing the soft tissue toward the bone mass in a controlled manner. It should be appreciated the ability to pull the first and second loop members 242, 244 relative to the suture strand loop 222 is permitted as a result of the fact that the engagement point for the first and second loop members 242, 244 and the suture strand loop 222 sits above the bone hole and there is not impediments of the free movement of the first and second loop members 242, 244 relative to the suture strand loop 222.

In particular, the first and second ends 246, 252 are pulled in a direction away from the first and second loop members 242, 244 causing the suture 234 to be drawn through the adjustable self-locking device 232 reducing the size of the first and second loop members 242, 244, and also tensioning the adjustable self-locking device 232 to lock the suture portions 262, 264 therein, and consequently drawing the soft tissue toward the bone mass since the size of the suture strand loop 222 is fixed. The first and second ends 246, 252 of the suture 234 are pulled and the sizes of the first and second loop members 242, 244 are reduced until such a time that the soft tissue is fully pulled toward the bone mass. The procedure may be repeated depending upon the needs of the procedure. The excess suture material of the first and second ends 246, 252 of the suture 234 may then be cut away and the incision closed.

As with the embodiment disclosed with reference to FIGS. 1 to 4, the above embodiment may be combined with an anchor sleeve as may be determined by the medical practitioner making using the present system.

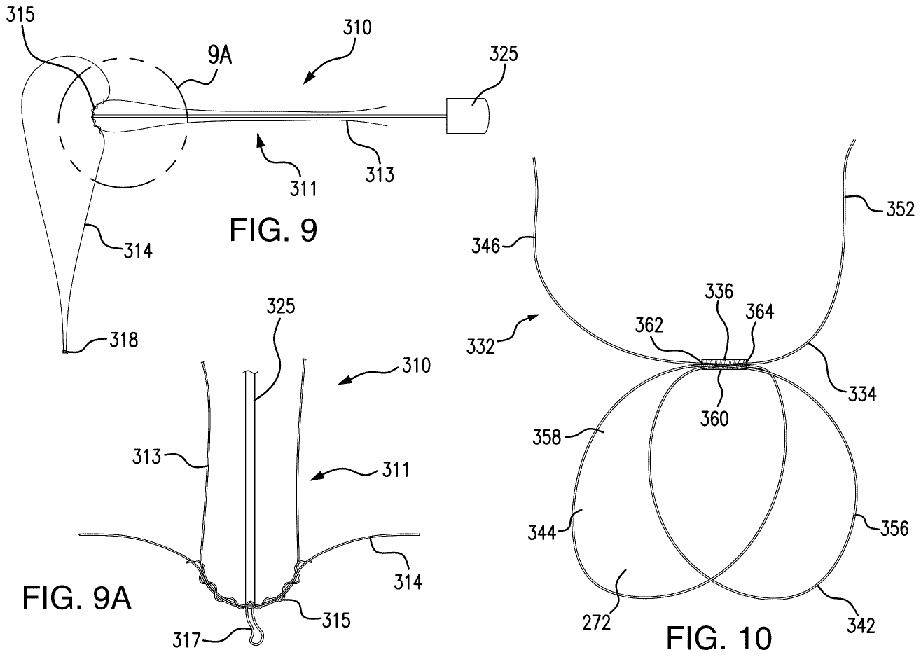

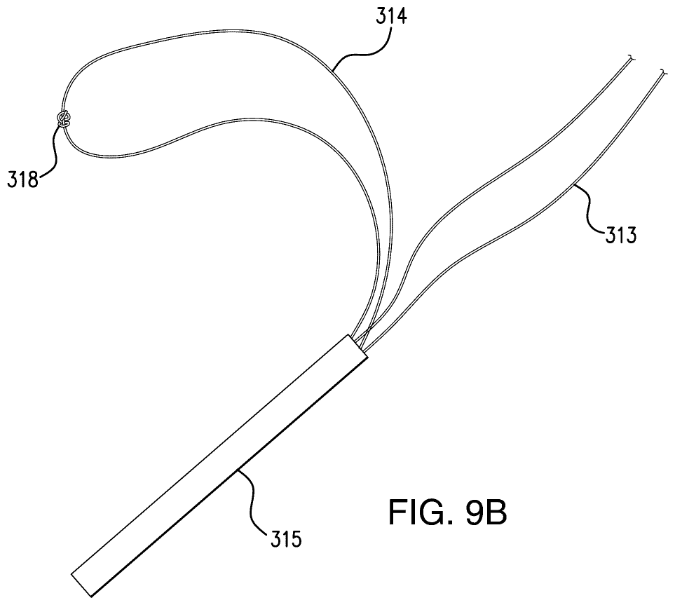

In accordance with yet another embodiment as shown with reference to FIGS. 9 to 15, the all-suture anchor assembly 310 further includes a suture capture assembly that is integrated onto with the all-suture anchor 311, thereby obviating the need for the capture members as discussed above with reference to FIGS. 1 to 8.

The all-suture anchor assembly 310 includes the all-suture anchor 311 that is composed of at least one suture strand 313, the all-suture anchoring element 315, and a suture strand loop 314. In accordance with a disclosed embodiment, the suture strand loop 314 is formed by creating a knot 318 (or other enlargement) with the ends of the suture forming the suture strand loop 314. As will be appreciated based upon the following disclosure, knot 318 (or some enlargement) allows the suture strand loop 314 to be captured and fixed distal to the deployed all-suture anchor 311 such that the knot 318 (or enlargement) is be fixed within the bone. The all-suture anchor assembly 310 also includes an all-suture anchoring element 315 threaded along the suture strand 313 in a manner discussed below in greater detail. The all-suture anchor assembly 310 also includes a suture loop capture assembly 317 integrated with the delivery inserter 325 for controlled capture of various elements in a manner discussed below in greater detail.

The all-suture anchoring element 315 is preferably composed solely of an enlarged piece of cylindrical suture material, a suture tape, or a suture sock (as described above). As with the prior embodiments, the enlarged surface area of the all-suture anchoring element 315 allows for the passage of the suture strand 313 therethrough in a manner providing for entanglement of the all-suture anchoring element 315 and the suture strand 313. The enlarged surface area of the all-suture anchoring element 315 also allows for the passage of the suture strand loop 314 therethrough in a manner providing for entanglement and the creating of a locked loop for purposes that will be described below in greater detail. The suture anchoring element 315 includes a first end and a second end, as well as a thickness, a width, and a length along a longitudinal axis. As briefly mentioned above, the all-suture anchor assembly 310 includes the suture strand 313, which is passed through, or weaved through, the all-suture anchoring element 315 at various locations along the length of the all-suture anchoring element 315 (so as to define the all-suture anchor 311). That is, the suture strand 313 penetrates and traverses the all-suture anchoring element 315 to define apertures in the all-suture anchoring element 315.

While the suture strand 313 is disclosed above as being threaded through the all-suture anchoring element 315, it is appreciated that the suture strand could be passed through a central passageway defined by the all-suture anchoring element where the all-suture anchoring element is constructed as an elongated cylindrical member. Similar, and while the embodiment above discloses the all-suture anchoring element 315 as having separate ends, the ends of the all-suture anchoring element could certainly be connected as is known in the art.

With the foregoing in mind, it is appreciated that a large variety of constructions and materials will work for the all-suture anchoring element. For example, the all-suture anchor may take a variety of forms as known in the art, for example, ConMed Y-Knot® & Y-Knot® RC, Parcus Draw Tight™, Smith & Nephew Suturefix Ultra, Smith & Nephew Q-FIX®, Zimmer Biomet: JuggerKnot® Soft Anchor; Cayenne Medical (Zimmer Biomet company) SureLock®, and Stryker® Iconix.

The all-suture anchor assembly 310 further includes the suture strand loop 314 and a suture capture assembly 317 integrated into the tip of the delivery inserter 325 for creation of a loop as described below in detail.

The delivery inserter 325 is operated by a surgeon whereby the surgeon manipulates the all-suture anchoring element 315, the suture strand 313, the suture strand loop 314, and the suture capture assembly 317 of the delivery inserter. With the suture stand loop 314, in particular, the knot 318 of the suture strand loop 314, coupled to the suture capture assembly 317 of the delivery inserter, the surgeon inserts the suture strand loop 314, the suture capture assembly 317, the all-suture anchoring element 315, and the entangled portion of the suture strand 313 in the bone hole by pushing the distal second end of the delivery inserter 325 into the bone hole. At that point, the surgeon can push a button or turn a device on the delivery inserter 325 which enables the deployment of the all-suture anchoring element 315. The all-suture anchoring element 315 is then folded, bent, creased, crinkled, bunched, or otherwise changed in shape after it is inserted into the bone hole in a manner that increases the size of the all-suture anchoring element 315 in a manner known to those skilled in the art and as described above.

The suture strand 313, the suture strand loop 314, the all-suture anchoring element 315, and the suture capture assembly 317 are installed within the bone hole in an elongated fashion to take advantage of a small diameter configuration, referred to herein as an undeployed state or installation state. Deployment occurs after the delivery inserter 325 is manipulated such that the capture assembly 317 captures and retains the knot 318 at one end of the suture strand loop 314. The suture strand loop 314 is retained in the bone due to the knot 318 (or some enlargement in the loop 314) that is captured and, therefore, maintained distal to the deployed all-suture anchoring element 315. This capture and retention are followed by positioning of the suture strand 313, the suture strand loop 314, the all-suture anchoring element 315, and the knot 318 within a bone hole for retention of the various elements to a bone mass.

In particular, the all-suture anchor 311 is deployed causing the all-suture anchoring element 315 to fold, bend, crease, crinkle, bunch or otherwise change shape in a manner that compresses the all-suture anchoring element 315. It is appreciated that deployment may occur before, after, or as the delivery inserter 325 is removed. As the all-suture anchoring element 315 is compressed it ultimately increases in size in a direction substantially perpendicular to the longitudinal axis of the bone hole (or otherwise oriented to contact side walls of the bone hole) and develops an outwardly directed force that is directed at the walls of the bone hole. As such, and after the delivery inserter 325, or other delivery instrument, has been removed, the all-suture anchoring element 315 exhibits an outward bias resulting in expansion of the all-suture anchoring element 315 in a direction substantially perpendicular to the longitudinal axis of the bone hole (or otherwise oriented to contact side walls of the bone hole) into which it is positioned, resulting in frictional engagement or gripping the wall of the bone, which is referred to herein as the expanded deployed state. As explained above, this increase in size holds the restrained end of the suture strand loop 314 within the bone hole with a portion of the suture strand loop sitting outside of the bone hole. It should also be noted the knot 318 or expansion on the suture strand loop 314 will aid to restrain the end of the suture strand loop 314 within the bone hole by increasing the force required for the suture strand loop 314 to be withdrawn past the deployed all-suture anchoring element 315.

As with the prior embodiment, the present invention achieves secure, tensioned attachment of soft tissue to a bone mass using the all-suture anchor assembly 310 as described above in conjunction with an adjustable self-locking device 332 composed of a length of suture 334 passing through a nonadjustable clinching portion 336 in such a way that first and second suture loop members 342, 344 are created. The adjustable self-locking device 332 is the same as that disclosed above with reference to FIGS. 1 to 4.

With reference to FIG. 10, the adjustable self-locking device 332 of this embodiment is preformed to include a double loop configuration having first and second loop members 342, 344 that each traverse a path from one end of the nonadjustable clinching portion 336 to the other end thereof. The adjustable self-locking device 332 is formed by passing the first end 346 of the suture through the nonadjustable clinching portion 336 and similarly passing the second end 352 through the nonadjustable clinching portion 336. In various aspects, the passageway defined by the nonadjustable clinching portion 336 is formed during a braiding process.

Passing ends 346, 352 through the passageway defined by the nonadjustable clinching portion 336 forms the first and second loop members 342, 344. The first and second loop members 342, 344 define mounting portions 356, 358 of the adjustable self-locking device 332 and can be disposed generally opposite from the nonadjustable clinching portion 336.

The longitudinal and parallel placement of the first and second ends 346, 352 of the suture 334 within the nonadjustable clinching portion 336 resists the reverse relative movement of the first and second ends 346, 352 of the adjustable self-locking device 332 once it is tightened. The tensioning of the first and second ends 346, 352 causes reciprocal movement of the first and second loop members 342, 344 relative to nonadjustable clinching portion 336. Upon applying tension to the first and second ends 346, 352, the first and second loop members 342, 344 are reduced to a desired size or placed in a desired tension. Tension in the first and second loop members 342, 344 causes the body of the suture defining the nonadjustable clinching portion 336 to be placed in tension and therefore cause the nonadjustable clinching portion 336 to constrict about the portions 362, 364 of the suture 334 passing through the passageway defined by the nonadjustable clinching portion 336. This constriction causes the adjustable self-locking device 332 to “automatically” lock in a reduced size or smaller diameter configuration.

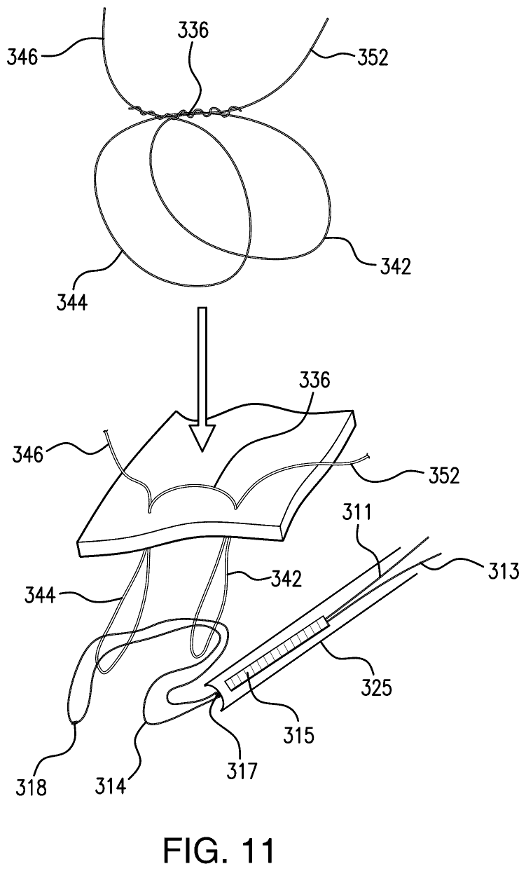

In accordance with the present method, and with reference to FIGS. 11 to 15, the adjustable self-locking device 332 is first passed through the soft tissue one wishes to secure to a bone mass. Referring to FIG. 11, the adjustable self-locking device 332, in particular, the first loop member 342 thereof is drawn through the soft tissue with a surgical needle. The second loop member 344 is passed through the soft tissue. In particular, the first loop member 342 and second loop member 344 are pulled through the soft tissue until the two mounting portions 356, 358 defined by the first and second loop members 342, 344 are of substantially the same size and in alignment. As shown in FIGS. 11, the first and second end 346, 352 of the suture 334, which function as tension members as will be discussed below in greater detail, of the adjustable self-locking device 332 also extends from one of the first and second loop members 342, 344 and are similarly accessible from outside of the joint under repair.

With the first and second loop members 342, 344 of the adjustable self-locking device 332 outside of the joint, and with reference to FIG. 11, the suture strand loop 314 (and knot 318 (or enlargement) on the suture strand loop 314) is passed through the openings 370, 372 respectively defined by the opposed first and second loop members 342, 344 such that the suture stand loop 314 is intertwined or linked with the opposed first and second loop members 342, 324. In this arrangement, the nonadjustable clinching portion 336 of the adjustable self-locking device 332 between the first and second loop members 342, 344 is in direct contact with the soft tissue securing the suture strand loop 314 to the soft tissue such that the first and second loop members 342, 344 may simultaneously pull against the suture strand loop 314 without fear that the adjustable self-locking device 332 will become disengaged with the soft tissue.

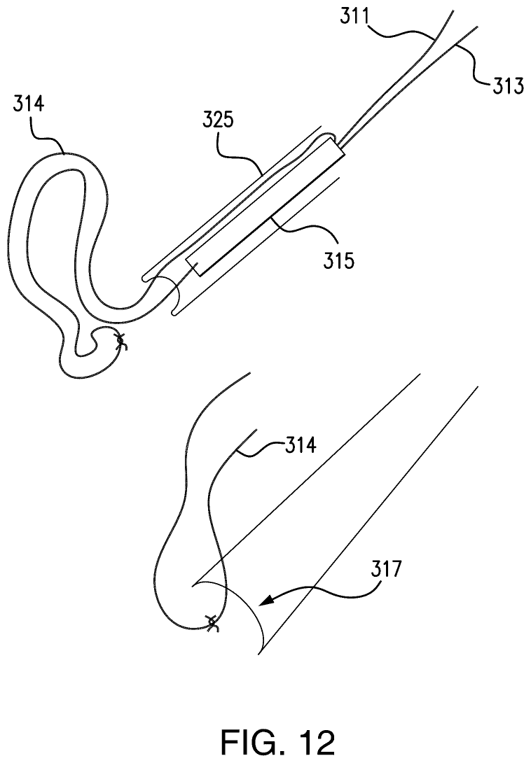

Thereafter, and with reference to FIG. 12, the loop end of the suture strand loop 314 is captured (preferably the knot 318 (or expansion) that creates the loop end of the suture strand loop 314 is captured by the capture assembly 317 of the delivery inserter 325); that is, the loop end of the suture strand loop 314 is captured, entangled, coupled to, or otherwise attached to the capture assembly 317 of the delivery inserter 325 for manipulation of the suture strand loop 314 and ultimately fixed attachment of the loop end of the suture strand loop 314 within the bone hole upon deployment of the all-suture anchor assembly 310 within the bone mass to which the soft tissue is secured. The delivery inserter 325, with the attached capture assembly 317 of the delivery inserter 325, are removed before, during, or after deployment of the all-suture anchor assembly 311 within the bone mass. The knot 318 that is used to create the suture strand loop 314 also provides increased resistance and maintains the suture strand loop 314 distal to the deployed all-suture anchoring element 315 and, therefore, securely within the bone mass.

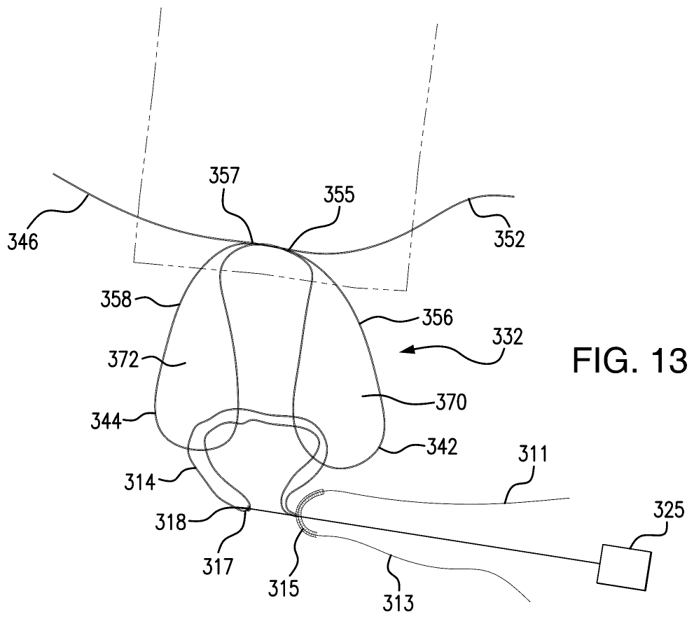

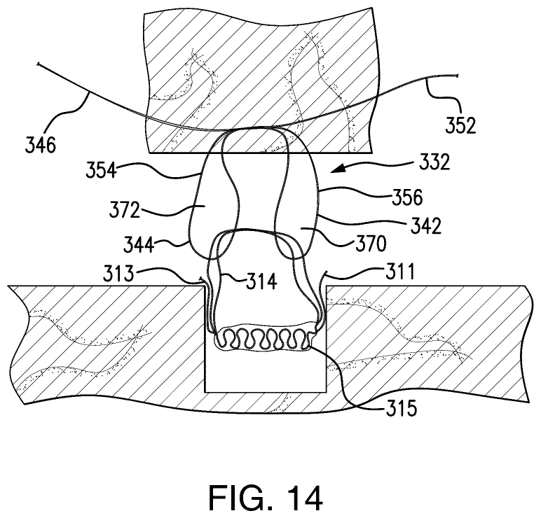

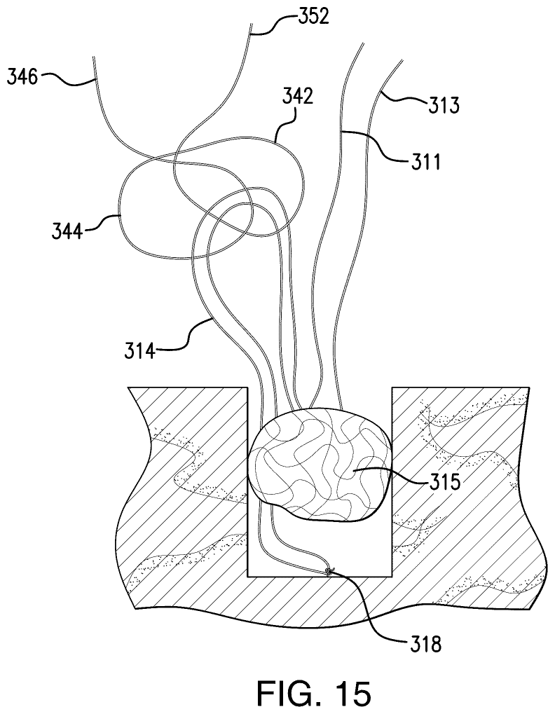

Referring to FIGS. 13, with the suture strand loop 314 passed through the openings 370, 372 defined by the opposed first and second loop members 342, 344 and the loop end of the suture strand loop 314 captured by capture assembly 317 of the delivery inserter 325, the suture strand loop 314 is linked to the first and second loop members 342, 344 and ultimately the soft tissue. Referring to FIGS. 14 and 15, the all-suture anchor assembly 310 is then inserted into the bone mass, either directly or within a predrilled anchor hole in the bone mass. The provision of the nonadjustable clinching portion 336 with the suture portions 362, 364 therethrough and under the control of the first and second ends 346, 352 of the suture 334, adjustment in the size of the first and second loop members 342, 344 provided for. This adjustability is useful in several aspects of the surgical procedure.

With the all-suture anchor assembly 310 securely held within the drill hole, the first and second ends 346, 352 of the suture 334 are pulled in a manner reducing the sizes of the first and second loop members 342, 344, and also tensioning the adjustable self-locking device 332, thereby drawing the soft tissue toward the bone mass in a controlled manner. It should be appreciated the ability to pull the first and second loop members 342, 344 relative to the suture strand loop 314 is permitted as a result of the fact that the engagement point for the first and second loop members 242, 244 and the suture strand loop 314 sits above the deployed all-suture anchoring element 315 or above the bone hole and there are not impediments of the free movement of the first and second loop members 342, 344 relative to the suture strand loop 314.

In particular, the first and second ends 346, 352 are pulled in a direction away from the first and second loop members 342, 344 causing the suture 334 to be drawn through the adjustable self-locking device 332 reducing the size of the first and second loop members 342, 344, and also tensioning the adjustable self-locking device 332 to lock the suture portions 362, 364 therein, and consequently drawing the soft tissue toward the bone mass since the size of the suture strand loop 322 is fixed. The first and second ends 346, 352 of the suture 334 are pulled and the sizes of the first and second loop members 342, 344 are reduced until such a time that the soft tissue is fully pulled toward the bone mass. The procedure may be repeated depending upon the needs of the procedure. The excess suture material of the first and second ends 346, 352 of the suture 334 may then be cut away and the incision closed.

The system for secure attachment of tissue to bone and other anatomical structure of the present invention as disclosed above with reference to the various embodiments, allows for expanded possibilities in surgical procedures. For example, the system of the present invention is well suited to create a horizontal mattress repair in the soft tissue. In addition, and while the various embodiments disclose an adjustable self-locking device with first and second loop members it is appreciated a plurality of adjustable self-locking devices may be used in combination or an adjustable self-locking device with more than two loop members may be used.

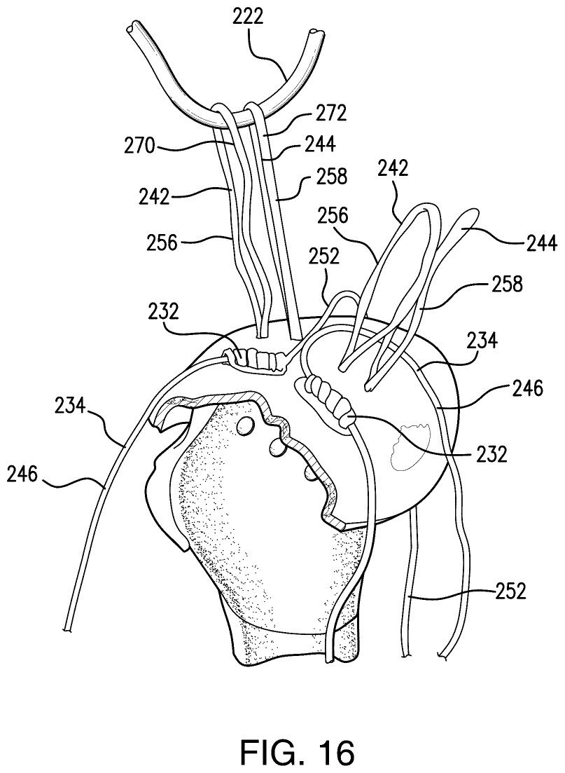

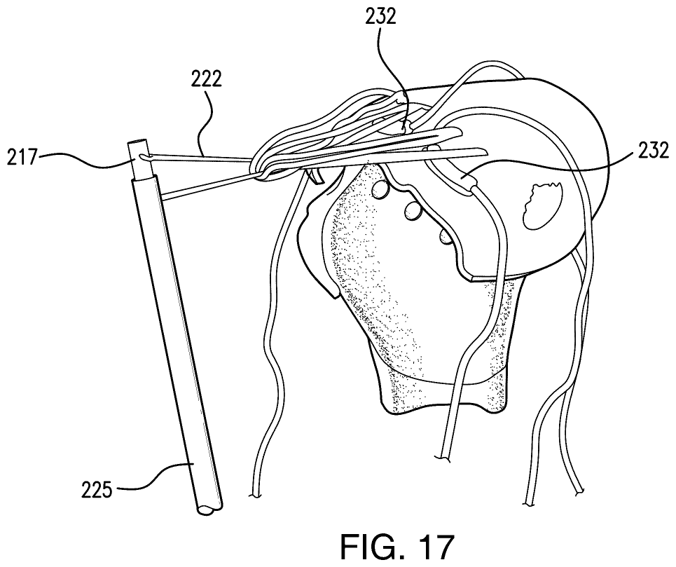

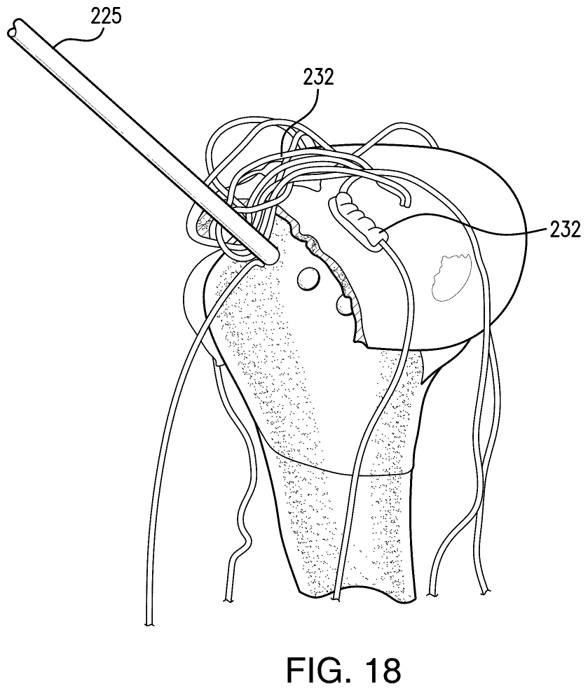

With reference to FIGS. 16, 17, and 18, the present system is used to create a “locking mattress repair” or “ripstop technique.” This repair procedure is disclosed below with reference to the embodiment disclosed with reference to FIGS. 6 to 8, although it is appreciated any of the embodiments disclosed herein may be used in conjunction with this methodology.

First and second adjustable self-locking devices 232 are first passed through the soft tissue one wishes to secure to a bone mass. In particular, the first loop members 242 thereof are drawn through the soft tissue with a surgical needle or a suture passing loop. The second loop members 244 are passed through the soft tissue. In particular, the first loop members 242 and second loop members 244 are pulled through the soft tissue until the two mounting portions 256, 258 defined by the first and second loop members 242, 244 are of substantially the same size and in alignment. The first and second ends 246, 252 of the suture 234, which function as tension members as will be discussed below in greater detail, of the adjustable self-locking device 232 also extends from one of the first and second loop members 242, 244 and are similarly accessible from outside of the joint under repair.

The first and second loop members 242, 244 are then again drawn through the soft tissue with a surgical needle at a position removed from the edge of the soft tissue such that the first and second loop member 242, 244 are on the same side of the tissue (that is, the top side for the sake of describing the present procedure) as the clinching portion 236.

With the first and second loop members 242, 244 of the adjustable self-locking device 232 outside of the joint and extending above the top side of the soft tissue at a position remote from the edge of the soft tissue, the suture strand loop 222 is passed through the openings 270, 272 respectively defined by the opposed first and second loop members 242, 244. That is, the free end of the suture strand loop 222 is drawn through the openings 270, 272 such that the suture stand loop 222 is intertwined or linked with the opposed first and second loop members 242, 224. In this arrangement, the clinching portion 236 of the adjustable self-locking device 232 between the first and second loop members 242, 244 is in direct contact with the soft tissue securing the suture strand loop 222 to the soft tissue such that the first and second loop members 242, 244 may simultaneously pull against the suture strand loop 222 without fear that the adjustable self-locking device 232 will become disengaged with the soft tissue.

Thereafter, the free end of the suture strand loop 222 is captured; that is, the free end of the suture strand loop 222 is captured, entangled, coupled to, or otherwise attached to the capture member 217 for manipulation of the suture strand loop 222 and ultimately fixed attachment of the free end of the suture strand loop 222 within the bone hole upon deployment of the all-suture anchor assembly 210 within the bone mass to which the soft tissue is secured.

With the suture strand loop passed through the openings 270, 272 defined by the opposed first and second loop members 242, 244 and the free end of the suture strand loop 222 captured by the capture member 217, the suture strand loop 222 is linked to the first and second loop members 242, 244 and ultimately the soft tissue. Referring to FIG. 13, the first and second loop members 242, 244 are pulled over the clinching portion 236 and the all-suture anchor assembly 210 is inserted into the bone mass, or within an anchor hole predrilled in the bone mass. The ‘rip-stop’ configuration is created by the first and second loop members 242, 244 being pulled over the clinching portion 236 and provides additional security of suture at the site of the soft tissue repair. The provision of the clinching portion 236 with the suture portions 262, 264 therethrough and under the control of the first and second ends 246, 252 of the suture 234, adjustment in the size of the first and second loop members 242, 244 is provided for. This adjustability is useful in several aspects of the surgical procedure.

With the all-suture anchor assembly 210 securely held within the drill hole, the first and second ends 246, 252 of the suture 234 are pulled in a manner reducing the sizes of the first and second loop members 242, 244, and also tensioning the adjustable self-locking device 232, thereby drawing the soft tissue toward the bone mass in a controlled manner. It should be appreciated the ability to reduce the sizes of the first and second loop members 242, 244 is permitted as a result of the fact that the engagement point for the first and second loop members 242, 244 and the suture strand loop 222 sits above the deployed all-suture anchoring element 215, or above the bone hole, and there are not impediments of the free movement of the first and second loop members 242, 244 relative to the suture capture member 217.

In particular, the first and second ends 246, 252 are pulled in a direction away from the first and second loop members 242, 244 causing the suture 234 to be drawn through the adjustable self-locking device 232 reducing the size of the first and second loop members 242, 244, and also tensioning the adjustable self-locking device 232 to lock the suture portions 262, 264 therein, and consequently drawing the soft tissue toward the bone mass since the size of the suture strand loop 222 is fixed. The first and second ends 246, 252 of the suture 234 are pulled and the first and second loop members 242, 244 size is reduced until such a time that the soft tissue is fully pulled toward the bone mass. The excess suture material of the first and second ends 346, 352 of the suture 334 may then be cut away and the incision closed.

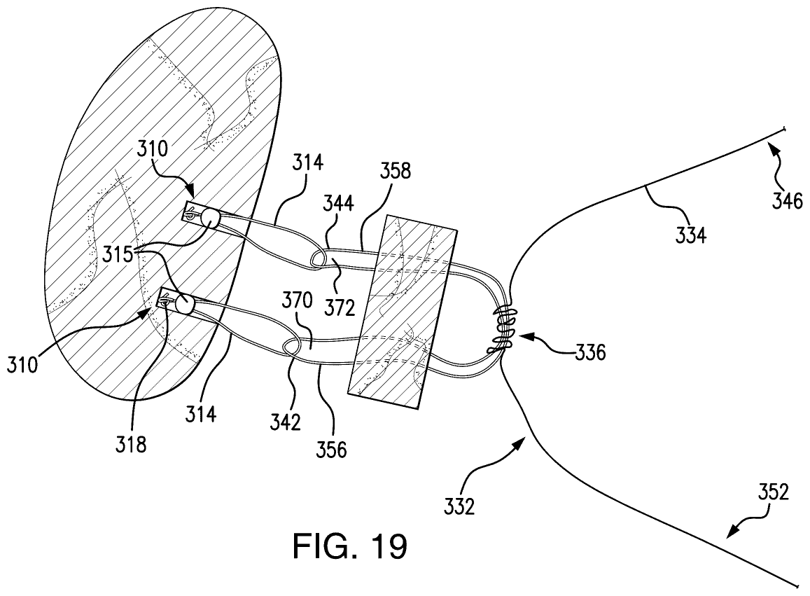

With reference to FIG. 19, the present system is used in a glenoid bone grafting procedure. This repair procedure is disclosed below with reference to the embodiment disclosed with reference to FIGS. 9 to 15, although it is appreciated any of the embodiments disclosed herein may be used in conjunction with this methodology.

An adjustable self-locking device 332 is first passed through the bone graft one wishes to secure to the glenoid. In particular, the first loop members 342 thereof is drawn through the bone graft with a surgical needle or a suture passing loop. The second loop members 344 is passed through the bone graft. In particular, the first loop members 342 and second loop members 344 are pulled through the bone graft until the two mounting portions 356, 358 defined by the first and second loop members 342, 344 are of substantially the same size and in alignment. The first and second ends 346, 352 of the suture 334, which function as tension members as will be discussed below in greater detail, of the adjustable self-locking device 332 also extends from one of the first and second loop members 342, 344 and are similarly accessible from outside of the joint under repair.

With the first and second loop members 342, 344 of the adjustable self-locking device 332 outside of the joint, first and second all-suture anchor assemblies 310 are used to secured the first and second loop members 342, 344 to the glenoid. The suture strand loops 314 are respectively passed through the openings 370, 372 defined by the opposed first and second loop members 342, 344.

Thereafter, the knots 318 of the suture strand loops 314 are captured by the capture assembly 317 of the inserter 325 (see FIG. 13) for manipulation of the suture strand loops 314 and ultimately fixed attachment of the suture strand loops 314 within the bone hole upon deployment of the all-suture anchoring elements 315 of the all-suture anchor assemblies 310 within the glenoid. The inserters 325 and capture assemblies 317 have been removed after deployment of the all-suture anchor assemblies 310 and are, therefore, not shown in FIG. 19.

With the all-suture anchor assemblies 310 securely held within the drill hole, the first and second ends 346, 352 of the suture 334 are pulled in a manner reducing the sizes of the first and second loop members 342, 344, and also tensioning the adjustable self-locking device 332, thereby drawing the bone graft toward the glenoid in a controlled manner. It should be appreciated the ability to reduce the size of the first and second loop members 342, 344 is permitted as a result of the fact that the engagement point for the first and second loop members 342, 344 and the suture strand loops 314 sit above the deployed all-suture anchoring element 315, or above the bone hole, and there are not impediments to the free movement of the first and second loop members 342, 344 relative to the suture strand loop 314.

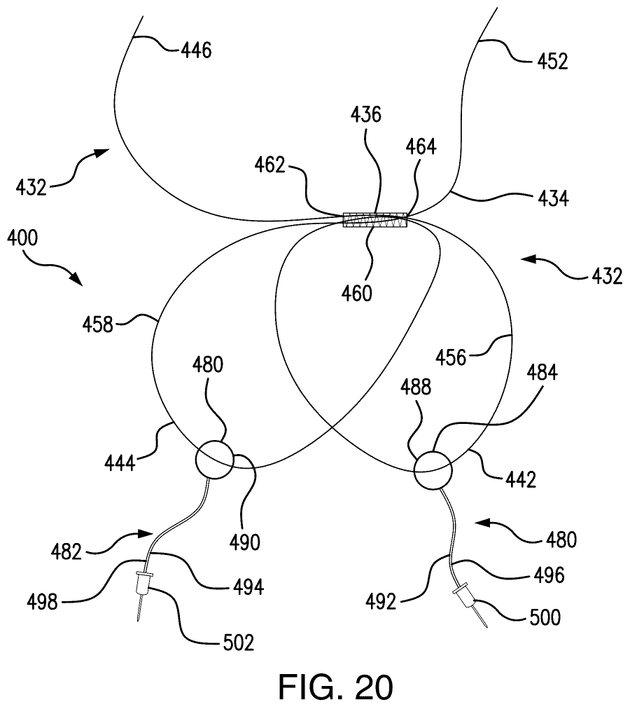

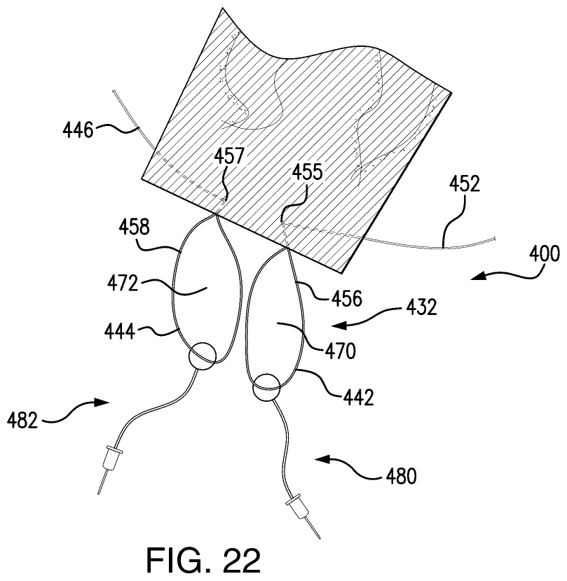

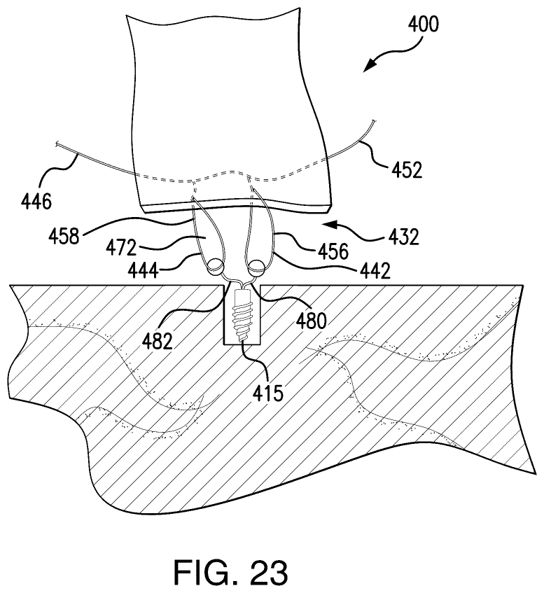

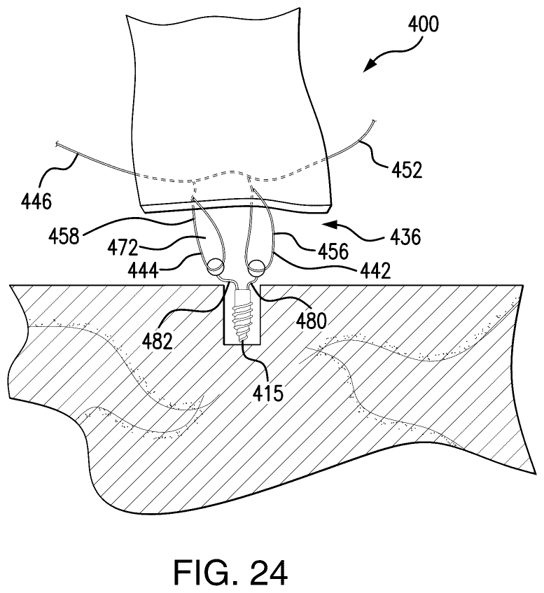

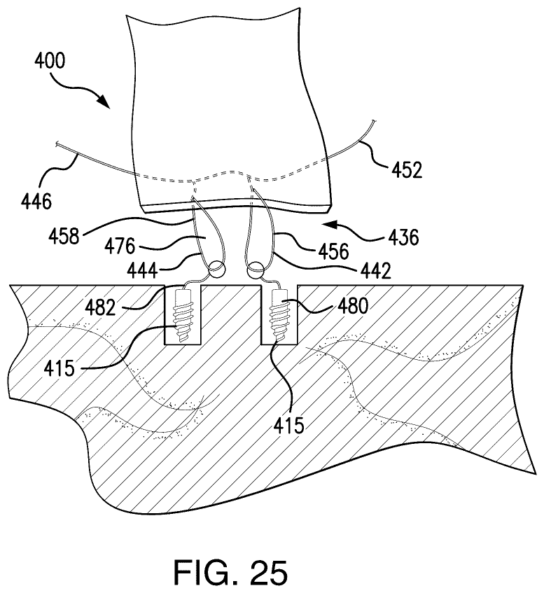

Referring to FIGS. 20 to 26, and in accordance with yet another embodiment, the system described above is modified by replacing the all-suture anchor assembly with passing sutures that are positioned along the first and second loop members of the preformed self-locking device. In particular, the system 400 includes an adjustable self-locking device 432 and first and second passing suture loops 480, 482 respectively secured to the respective first and second loop members 442, 444 of the adjustable self-locking device 432.

With reference to FIG. 20, a preformed adjustable self-locking device 432 is disclosed. The adjustable self-locking device 432 is preformed to include a double loop configuration having first and second loop members 442, 444 that each traverse a path from one end of the clinching portion 436 to the other end thereof. The adjustable self-locking device 432 is formed by passing the first end 446 of the suture through the clinching portion 436 and similarly passing the second end 452 through the clinching portion 436. In various aspects, the passageway defined by the clinching portion 436 is formed during a braiding process.

Passing ends 446, 452 through the passageway defined by the clinching portion 436 forms the first and second loop members 442, 444. The first and second loop members 442, 444 define mounting portions 456, 458 of the adjustable self-locking device 432 and can be disposed generally opposite from the clinching portion 436.

The longitudinal and parallel placement of the first and second ends 446, 452 of the suture 434 within the clinching portion 436 resists the reverse relative movement of the first and second ends 446, 452 of the adjustable self-locking device 432 once it is tightened. The tensioning of the first and second ends 446, 452 causes reciprocal movement of the first and second loop members 442, 444 relative to clinching portion 436. Upon applying tension to the first and second ends 446, 452, the first and second loop members 442, 444 are reduced to a desired size or placed in a desired tension. Tension in the first and second loop members 442, 444 causes the body of the suture defining the clinching portion 436 to be placed in tension and therefore cause the clinching portion 436 to constrict about the portions 462, 464 of the suture 434 passing through the passageway defined by the clinching portion 436. This constriction causes the adjustable self-locking device 432 to “automatically” lock in a reduced size or smaller diameter configuration for the two loop members 442, 444.

The adjustable self-locking device 432 also includes first and second passing suture loops 480, 482 respectively secured to the respective first and second loop members 442, 444. As those skilled in the art will appreciate, a passing suture loop 480, 482 is a suture member preformed with a loop 484, 486 at a first end 488, 490 thereof and a free suture strand 492, 494 at the second end 496, 498 thereof. A passing surgical needle 500, 502 is releasably secured at each of the distal ends 504, 506 of the second ends 496, 498 of the first and second passing suture loops 480. It is appreciated that a passing surgical needle is not required. For example, the free end of the passing suture loop can be passed through the soft tissue using an arthroscopic suture passer. Several commercially available, arthroscopic suture passers can engage with and pass a suture strand through soft tissue.

In accordance with present invention, the loop 484 of the first passing suture loop 480 is secured to the first loop member 442 with the suture material of each loop passing through the aperture defined by the other loop. Similarly, the loop 486 of the second passing suture loop 482 is secured to the second loop member 444 with the suture material of each loop passing through the aperture defined by the other loop.

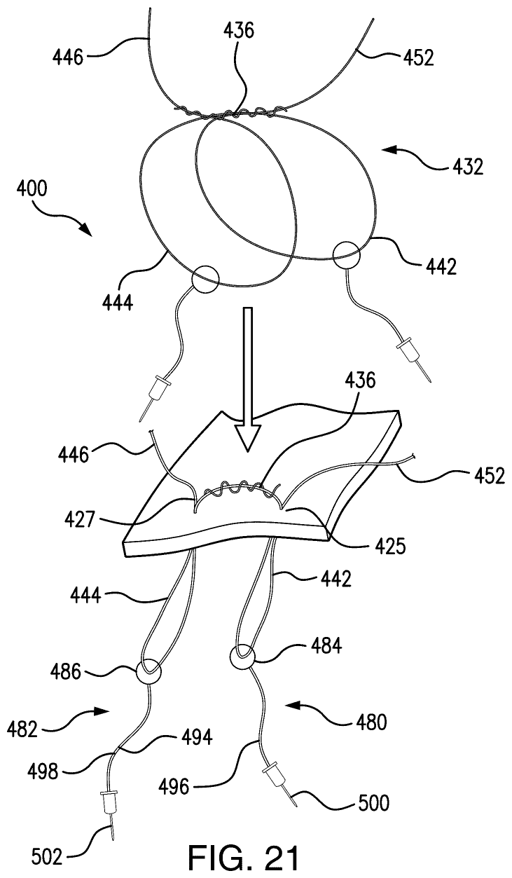

In accordance with the present method, the adjustable loops 442, 444 of the self-locking device 432 is first passed through the soft tissue one wishes to secure to a bone mass. Referring to FIG. 21, the adjustable self-locking device 432, in particular, the passing surgical needle 500, the first passing suture loop 480, and the first loop member 442 are drawn through the soft tissue with a surgical needle 500 at a first location 425. Similarly, the second surgical needle 502, the second passing suture loop 482, and the second loop member 444 are drawn through the soft tissue with a second surgical needle 502 at a second location 427. As mentioned above, the passing suture loops 480, 482 do not require an attached passing surgical needle and commercially available suture passers having a needle within the suture passer could be employed.

In particular, the first loop member 442 and second loop member 444 are pulled through the soft tissue until the two mounting portions 456, 458 defined by the first and second loop members 442, 444 are of substantially the same size (or different sizes if the application so requires) and in alignment, with the first and second passing suture loops 480, 482 hanging from the first and second loop members 442, 444 on the same side of the tissue. It is appreciated the first and second loop members 442, 444 are relatively long so that the opposed mounting portions 456, 458 may be brought outside of the joint under repair. The first and second end 446, 452 of the suture 434, which function as tension members as will be discussed below in greater detail, of the adjustable self-locking device 432 also extends from one of the first and second loop members 442, 444 and are similarly accessible from outside of the joint under repair.

With the first and second loop members 442, 444 of the adjustable self-locking device 432 outside of the joint, and with reference to FIGS. 21, 22, and 23, the free suture strand 492, 494 at the second end 496, 498 of the passing suture loops 480, 482 are sequentially captured by a bone anchor(s) 415 (any commonly available bone anchor may be used) and the bone anchor(s) 415 is seated within a bone hole(s) at a predetermined location. In accordance with one embodiment, the free suture strands 492, 494 of the passing suture loops 480, 482 may be positioned within the same bone hole using a single bone anchor 415 to engage the free suture strands 492, 494 of the passing suture loops 480, 482. In accordance with another embodiment, the free suture strands 492, 494 of the passing suture loops 480, 482 may be positioned within the same bone hole using a separate and distinct bone anchors 415 to respectively engage the free suture strands 492, 494 of the passing suture loops 480, 482. In accordance with yet another embodiment, the free suture strands 492, 494 of the passing suture loops 480, 482 may be positioned within the separate bone holes using distinct bone anchors 415 to engage the free suture strands 492, 494 of the passing suture loops 480, 482.

Regardless of which embodiment is selected, the free suture strands 492, 494 and bone anchors 415 are positioned within the bone holes such that the loops 484, 486 of the passing suture loops 480, 482 remain above the outer surface of the bone into which the bone anchor(s) 415 is positioned (or above the area of contact of bone anchor(s) 415 and the bone, if the passing suture loops 480, 482 are secured below the outer surface of the bone). As such, the first and second loops 442, 444 are allowed to move freely where they are linked to the passing suture loops 480, 482 when the bone anchor(s) 415 are anchored. It should be appreciated, one embodiment is disclosed herein and the passing suture loops 480, 482 don't necessarily need to ‘remain above the outer surface of the bone’. The passing suture loops 480, 482 can be recessed below the outer surface of the bone, as long as the loops 484, 486 of the passing suture loops 480, 482 remain above the area of contact of the bone anchor(s) 415 and the wall of the bone hole. Even if the passing suture loops 480, 482 are recessed below the outer surface of the bone, but above the area of contact of the bone anchor 415 and the bone hole, the adjustable loops 442, 444 can move freely in their linkage with passing suture loops 480, 482.

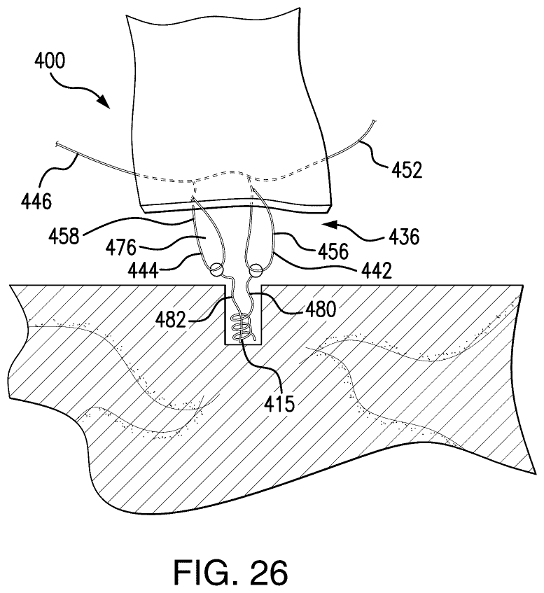

While a bone anchor 415 is disclosed above, it is appreciated an all-suture anchor 415s could replace the bone anchor 415. In such an embodiment, and with reference to FIGS. 21 and 26, the free second end 496, 498 of the suture passing loop 480, 482 is intertwined with the suture material of the all-suture anchor 415s and the all-suture anchor 415s is anchored within a bone hole.

Once the passing suture loops 480, 482 are anchored, the first and second ends 446, 452 of the suture 434 are pulled in a manner reducing the sizes of the first and second loop members 442, 444, and also tensioning the adjustable self-locking device 432, thereby drawing the soft tissue toward the bone mass in a controlled manner. The ability to reduce the size of the first and second loop members 442, 444 is permitted as a result of the fact that the suture-to-suture engagement point of the first and second loop members 442, 444, and the loop portions 484, 486 of the passing suture loops 480, 482 have no impediment to the free movement of the first and second loop members 442, 444 relative to the loop portions 484, 486 of the passing suture loops 480, 482.

In particular, the first and second ends 446, 452 are pulled in a direction away from the first and second loop members 442, 444 causing the suture 434 to be drawn through the adjustable self-locking device 432 reducing the size of the first and second loop members 442, 444, and also tensioning the adjustable self-locking device 432 to lock the suture portions 462, 464 therein, and consequently drawing the soft tissue toward the bone mass. The first and second ends 446, 452 of the suture 434 are pulled and the sizes of the first and second loop members 442, 444 are reduced until such a time that the soft tissue is fully pulled toward the bone mass. The procedure may be repeated to provide the desired degree of tension on the repair. The excess suture material of the first and second ends 446, 452 of the suture 434 may then be cut away after tensioning is complete. Alternatively, the first and second ends 446,452 can be anchored to the bone with commercially available suture anchors.

As those skilled in the art will certainly appreciate the soft tissue repair procedure described above may be extended to other soft tissue repairs (for example, labral repairs) and to bone grafting procedures. First and second loop 442, 444 can be passed through holes in a bone block or bone graft and secured to a bone surface in the same manner as described above for soft tissue repair. Referring to the various embodiments disclosed above, it is described that the excess suture material at the first and second ends (146, 152; 246, 252; 346, 352; 446, 452) of the adjustable self-locking device (132, 232, 332, 432) is cut away at the end of the procedure. Alternatively, a dual-row (or transosseous equivalent repair) can be created by anchoring these suture strands (146, 152; 246, 252; 346, 352; or 446, 452) to the bone at another location using a standard knotless suture anchor (such as the Arthrex SwiveLock anchor, Smith & Nephew Footprint Ultra PK Knotless Anchor, Smith & Nephew Healicoil Knotless, or Mitek Healix Advance Knotless Anchor) before cutting the excess suture material. In the case of the glenoid bone graft procedure, the excess suture material at the first and second ends (446, 452) can be passed through the labrum and/or glenohumeral ligament before these suture ends are secured to the glenoid, at another location, using a standard knotless suture anchor.

In many situations throughout the discussion above, the terminology relating to the secure attachment of soft tissue to bone mass has been used. Such terminology refers to the attachment or reattachment of tissue to a bone mass by securely binding the tissue to the bone mass utilizing the novel knotless suture anchor assembly. The suture element can be made up of a known suture material, or it can be made of polymer materials, or can be formed of bioabsorbable/biocomposite material such as a polylactide polymer.

While the preferred embodiments have been shown and described, it will be understood that there is no intent to limit the invention by such disclosure, but rather, is intended to cover all modifications and alternate constructions falling within the spirit and scope of the invention.

1. A method for secure attachment of tissue through application of a system comprising at least two suture passing loops and an adjustable self-locking device including a length of suture passing through at least one clinching portion to define a first loop member and a second loop member of the at least two passing loops, the method comprising:
passing the at least two suture passing loops and the adjustable self-locking device through soft tissue configured to be secured to a bone mass;
anchoring the at least one suture passing loop within a bone hole;
tensioning the adjustable self-locking device to draw the soft tissue in a controlled manner until such a time that the soft tissue is fully pulled into position.

2. The method according to claim 1, wherein the adjustable self-locking device includes a first suture passing loop linked to the first loop member and a second passing loop linked to the second loop member and the method includes anchoring the first suture passing loop and the second passing loop and at least two passing surgical needles releasably secured at each of distal ends of the first passing sure loop and the second passing suture loop.

3. The method according to claim 2, wherein a suture anchor is configured to anchor the suture passing loop to bone.

4. The method according to claim 2, wherein an all suture anchor is configured to anchor the suture passing loop to bone.

5. The method according to claim 1, wherein the bone anchor is configured to anchor the first and second suture passing loops.

6. The method according to claim 5, wherein the bone anchor is an all suture anchor.

7. The method according to claim 1, wherein passing the at least two suture passing loops and the adjustable self-locking device is configured to draw the first loop member and the second loop member of the adjustable self-locking device through the soft tissue.

8. The method according to claim 1, wherein tensioning the adjustable self-locking device includes pulling first and second ends of the length of suture linked to the first and second loop members in a direction away from the first and second loop members causing a length of suture to be drawn through the at least one clinching portion reducing a size of the first and second loop members and also tensioning the adjustable self-locking device to draw the soft tissue into position.

9. The method according to claim 1, further including placing the at least one clinching portion of the adjustable self-locking device between the first loop member and the second loop member in direct contact with the soft tissue.

10. A system for secure attachment of tissue to bone and other anatomical structures, comprising:
at least one free suture strand with at least two suture passing loops; and an adjustable self-locking device including a length of suture passing through at least one clinching portion so as to define at least one loop member, whereby the at least two suture passing loops of the at least one free suture strand remains above an area of contact enabling free movement of the at least two suture passing loops.

11. The system according to claim 10, wherein the at least two suture passing loops are secured to the at least one loop member of the adjustable self-locking device.

12. The system according to claim 11, further including a bone anchor that is configured to anchor the at least one suture passing loop to bone.

13. The system according to claim 11, further including an all suture anchor that is configured to anchor the at least one suture passing loop to bone.

14. The system according to claim 10, further including a bone anchor that is configured to anchor the first and second suture passing loops.

15. The system according to claim 10, further including an all suture anchor that is configured to anchor the first and second suture passing loops and two passing surgical needles releasably secured at each of distal ends the first passing suture loop and the second passing suture loop.
